(19) United States
(12) Patent Application Publication
(10) Pub. No.: US 2025/0319009 A1
(43) Pub. Date: Oct. 16, 2025

(54) PERSONAL CARE COMPOSITION SUBSTANTIALLY FREE OF ALKYL SULFATE OR ALKYL ETHER SULFATE TYPE OF SURFACTANTS

(71) Applicant: The Procter & Gamble Company, Cincinnati, OH (US)

(72) Inventors: Olubolaji Akintelure, Mason, OH (US); Wei Ji, Cincinnati, OH (US); Sean Michael Renock, Loveland, OH (US); Karl Shiqing Wei, Mason, OH (US)

(21) Appl. No.: 19/175,066

(22) Filed: Apr. 10, 2025

Related U.S. Application Data
(60) Provisional application No. 63/632,809, filed on Apr. 11, 2024.

(51) Int. Cl. A61K 8/44; A61K 8/06; A61K 8/37; A61K 8/39; A61K 8/73; A61K 8/92; A61Q 5/02; A61Q 19/10

(52) U.S. Cl. CPC A61K 8/442; A61K 8/062; A61K 8/375; A61K 8/39; A61K 8/732; A61K 8/922; A61Q 5/02; A61Q 19/10; A61K 2800/30; A61K 2800/5428; A61K 2800/5922; A61K 2800/805

(57) ABSTRACT

A personal care composition having at least a cleansing phase and a benefit phase. The composition is substantially free of alkyl sulfate and alkyl ether sulfate type of surfactants. The cleansing phase includes: an alaninate surfactant; a zwitterionic or amphoteric surfactant; and a structuring system. The structuring system has from 0.5% to 5% of an emulsifying agent by weight of the composition. The emulsifying agent is a glyceryl ester and/or a non-ionic emulsifier having an HLB of from 3.4 to 13.0. The glyceryl ester is selected from glyceryl laurate, glyceryl caprate, glyceryl caprylate, glyceryl caprylate/caprate, glyceryl stearate, and a mixture thereof. The non-ionic emulsifier having an HLB of from 3.4 to 13.0 includes trideceth-3 or trideceth-4. The structuring system also includes from 0.01% to 10% of a rheology modifier by weight of the composition. The benefit phase includes from 0.1% to 50% of a benefit agent by weight of the composition. The personal care composition has a pH that is from 4.0 to 5.5.

### FIELD OF THE INVENTION

The present disclosure generally relates to personal care compositions, which exhibit improved rheology, viscosity and lather properties, their methods and their uses. The present disclosure relates to personal care compositions, particularly to personal cleansing compositions for cleansing hair or skin. More specifically, a personal care composition includes at least a cleansing phase and a benefit phase, and the cleansing phase includes an alaninate surfactant; a zwitterionic or amphoteric surfactant; and a specific structuring system. The personal care composition pertains to a sulfate-substantially free surfactant system. The personal care composition is substantially free of alkyl sulfate or alkyl ether sulfate type of surfactants.

### BACKGROUND OF THE INVENTION

Cleansing the skin has been a common practice for centuries, with early cleansers relying on soap chemistry or mechanical action to remove dirt, sweat, sebum, and body odors. Soap-based cleansers and body washes have been widely used, along with various personal care compositions, to achieve effective skin cleansing.

Personal care compositions often require the inclusion of structuring agents to suspend and stabilize dispersions of benefit agents while maintaining the physical integrity of the composition. The ability to provide structure is crucial for the overall performance of personal care compositions. However, striking the right balance between structure and micellar formation upon dilution is a challenging task. Excessive structure can result in inferior performance, while inadequate structure may lead to instability of the composition.

In recent years, personal care compositions containing sodium trideceth-2 sulfate and a structuring system based on specific associative polymers have been explored. These compositions have shown promising results in terms of providing the desired structure and cleansing efficiency. However, sodium trideceth-2 sulfate is a sulfate-based surfactant. There is a growing demand for sulfate-free alternatives due to concerns regarding potential skin irritation and environmental impact.

Therefore, there is a need for a personal care composition that eliminates the use of sodium trideceth-2 sulfate while maintaining the desired rheology, viscosity and lather properties. Such a composition would address the demand for sulfate-free formulations while providing effective skin cleansing and deposition of benefit agents.

### SUMMARY OF THE INVENTION

A personal care composition is provided and comprises at least a cleansing phase and a benefit phase. The composition is substantially free of alkyl sulfate and alkyl ether sulfate type of surfactants. Optionally, the cleansing phase may comprise an aqueous structured surfactant phase. The cleansing phase comprises: an alaninate surfactant; a zwitterionic or amphoteric surfactant; and a structuring system. The structuring system comprises from about 0.5% to about 5% of an emulsifying agent by weight of the composition. The emulsifying agent is a glyceryl ester and/or a non-ionic emulsifier having an HLB of from 3.4 to 13.0. The glyceryl ester is selected from glyceryl laurate, glyceryl caprate, glyceryl caprylate, glyceryl caprylate/caprate, glyceryl stearate, and a mixture thereof. The non-ionic emulsifier having an HLB of from 3.4 to 13.0 comprises trideceth-3 or trideceth-4. The structuring system also comprises from about 0.01% to about 10% of a rheology modifier by weight of the composition. The benefit phase comprises from about 0.1% to about 50% of a benefit agent by weight of the composition. The personal care composition has a pH that is from about 4.0 to about 5.5, preferably from about 4.2 to about 5.3, more preferably from about 4.5 to about 5.25, most preferably from about 4.90 to about 5.25.

A method of making a liquid personal care composition having an acceptable lather or providing a substantial amount of foam or lather when agitated or mixed with water is provided and comprises the step of forming a personal care composition as described herein.

### DETAILED DESCRIPTION OF THE INVENTION

### Definitions of Terms

In this document, the following definitions apply unless specifically stated otherwise.

All percentages are by weight (w/w) of the composition, unless otherwise specified. “% wt.” means percentage by weight. References to ‘parts’ e.g. a mixture of 1 part X and 3 parts Y, is a ratio by weight. All ratios or percentages are weight ratios or weight percentages unless specifically stated otherwise.

An “active composition” is the composition absent water, and an “active ingredient” is the ingredient absent its water.

“QS” or “QSP” means sufficient quantity for 100% or for 100 g. +/− indicates the standard deviation. All ranges are inclusive and combinable. The number of significant digits conveys neither a limitation on the indicated amounts nor on the accuracy of the measurements. All numerical amounts are understood to be modified by the word “about”.

All measurements are understood to be made at 25° C. and at ambient conditions, where “ambient conditions” means at 1 atmosphere (atm) of pressure and at 65% relative humidity, unless otherwise stated. “Relative humidity” refers to the ratio (stated as a percent) of the moisture content of air compared to the saturated moisture level at the same temperature and pressure. Relative humidity can be measured with a hygrometer, in particular with a probe hygrometer from VWR® International.

Herein “min” means “minute” or “minutes”. Herein “mol” means mole. Herein “g” following a number means “gram” or “grams”. “Ex.” means “example”. All amounts as they pertain to listed ingredients are based on the active level and do not include carriers or by-products that may be included in commercially available materials.

Herein, “comprising” means that other steps and other ingredients can be in addition. “Comprising” encompasses the terms “consisting of” and “consisting essentially of”. The compositions, methods, uses, and processes described herein can comprise, consist of, and consist essentially of the elements and limitations described herein, as well as any of the additional or optional ingredients, components, steps, or limitations described herein. Embodiments and aspects described herein may comprise or be combinable with elements, features or components of other embodiments and/or aspects despite not being expressly exemplified in combination, unless an incompatibility is stated.

As used herein, the articles including “a” and “an” when used in a claim, are understood to mean “one or more” of what is claimed or described.

The terms “include,” “includes,” and “including,” as used herein are meant to be non-limiting.

Where amount ranges are given, these are to be understood as being the total amount of said ingredient in the composition, or where more than one species fall within the scope of the ingredient definition, the total amount of all ingredients fitting that definition, in the composition.

For example, if the composition comprises from 1% to 5% fatty alcohol, then a composition comprising 2% stearyl alcohol and 1% cetyl alcohol and no other fatty alcohol, would fall within this scope.

The amount of each particular ingredient or mixtures thereof described hereinafter can account for up to 100% (or 100%) of the total amount of the ingredient(s) in the composition.

The term “free of” as used herein means that the composition comprises 0% of an ingredient by weight of the composition, thus no detectable amount of the stated ingredient.

The term “substantially free of” as used herein means less than about 1.5%, less than about 1.2%, less than about 1%, less than about 0.8%, less than about 0.5%, less than about 0.3%, less than about 0.1%, less than about 0.01% or less than an immaterial amount of by weight of the composition.

Herein “Comp. Ex.” or “C. Ex.” means comparative example; and “Ex.” means example.

The term “molecular weight” or “M.Wt.” as used herein refers to the weight average molecular weight unless otherwise stated. The weight average molecular weight can be measured by gel permeation chromatography (“GPC”).

The term “personal care composition” as used herein, refers to compositions intended for topical application to the skin, hair, or scalp. The compositions described herein are rinse-off formulations, in which the product is applied topically to the skin, hair, or scalp and then is subsequently rinsed within minutes from the skin or hair or scalp with water, or otherwise wiped off using a substrate with deposition of a portion of the composition. The compositions also may be used as shaving aids. The personal care composition is typically extrudable or dispensible from a package. The personal care compositions typically exhibit a Carreau zero shear viscosity of from about 200 Pa·s (200,000 centipoise (cP)) to about 16 000 Pa·s (16,000,000 cP); or from about 500 Pa·s (500,000 centipoise (cP)) to about 16 000 Pa·s (16,000,000 cP); or from about 500 Pa·s (500,000 centipoise (cP)) to about 13 000 Pa·s (13,000,000 cP), or from about 500 Pa·s (500,000 centipoise (cP)) to about 7 750 Pa·s (7,750,000 cP); or from about 1 500 Pa·s (1,500,000 centipoise (cP)) to about 16 000 Pa·s (16,000,000 cP) as measured by the Carreau Zero Shear Viscosity Method as disclosed herein. The personal care compositions can be in the form of liquid, semi-liquid, cream, lotion or gel compositions intended for topical application to skin. Examples of personal care compositions can include but are not limited to shampoo, conditioning shampoo, body wash, moisturizing body wash, shower gels, skin cleansers, cleansing milks, hair and body wash, in shower body moisturizer, pet shampoo, shaving preparations and cleansing compositions used in conjunction with a disposable cleansing cloth.

The term “personal cleansing composition” as used herein refers to compositions intended for topical application to the hair and the skin, preferably to the skin, for cleansing.

The term “mixtures” as used herein is meant to include a simple combination of materials and any compounds that may result from their combination.

The term “room temperature” refers to a temperature of 25° C.

The term “rinse-off” as used herein means the intended product usage includes application to skin followed by rinsing and/or wiping the product from the skin within a few seconds to minutes of the application step. The product is generally applied and rinsed in the same usage event, for example, a shower or washing one's hands.

The term “derivative” as used herein refers to structures which are not shown but which one skilled in the art would understand are variations of the basic compound.

The term “structured,” as used herein means having a rheology that confers stability on the personal care composition. The personal care composition having at least a cleansing phase and a benefit phase may be defined as a multiphase composition. The degree of structure is determined by characteristics determined by one or more of the following methods: The Carreau Zero Shear Viscosity Method or by the Ultracentrifugation Method, all in the Test Methods below. Accordingly, a cleansing phase of the personal care composition or the personal care composition is considered “structured,” if the surfactant cleansing phase or the personal care composition has one or more of the following properties described below according to the Carreau Zero Shear Viscosity Method or by the Ultracentrifugation Method. A surfactant phase is considered to be structured, if the phase has one or more of the following characteristics:
A. a Carreau Zero Shear Viscosity from about 200 Pa·s to about 16 000 Pa·s, preferably from about 500 Pa·s to about 16 000 Pa·s, or from about 500 Pa·s to about 13 000 Pa·s, more preferably from about 1000 Pa·s to about 12000 Pa·s, even more preferably from about 2900 Pa·s to about 11775 Pa·s, most preferably from about 4500 Pa·s to about 11600 Pa·s; or from about 500 Pa·s to about 7750 Pa·s; or from about 1 500 Pa·s to about 16 000 Pa·s as measured by the Carreau Zero Shear Viscosity Method as disclosed herein; or
B. a Structured Domain Volume Ratio as measured by the Ultracentrifugation Method described hereafter, of greater than about 40%, or at least about 50%, or at least about 55%, or at least about 60 or at least about 65%, or at least about 70%, or at least about 75%, or at least about 80%, or at least about 85%, or at least about 90%.

The term “lather” as used herein, means an aerated foam which results from providing energy to aqueous surfactant mixtures, especially dilute mixtures. Lather is increased in micellar compositions compared to structured, e.g., lamellar compositions, so that a phase change during dilution to micelles typically increases lather.

The term “visually distinct” as used herein, refers to a region of the personal care composition having one average composition, as distinct from another region having a different average composition, wherein the regions are visible to the unaided naked eye. This would not preclude the distinct regions from comprising two similar phases where one phase could comprise pigments, dyes, particles, and various optional ingredients, hence a region of a different average composition. A phase generally occupies a space or spaces having dimensions larger than the colloidal or sub-colloidal components it comprises. A phase can also be constituted or re-constituted, collected, or separated into a bulk phase in order to observe its properties, e.g., by centrifugation, filtration or the like.

The methods as disclosed herein are cosmetic methods or non-therapeutic methods.

The personal care compositions, methods and uses of the compositions, the structures and the respective compositions as described in the Summary or as described hereinbelow are for fulfilling the technical effects or goals as set out herein. These objects and other advantages as may be apparent to those skilled in the art can be achieved through the personal care compositions, methods and uses of the compositions as described herein.

### Benefits

The present disclosure offers a personal care composition that overcomes the limitations of existing formulations. The composition comprises a sulfate-free surfactant system, which provides improved rheology, viscosity and lather properties compared to conventional sulfate-based systems.

By eliminating sodium trideceth-2 sulfate, the personal care composition reduces the risk of skin irritation and environmental impact associated with sulfate-based surfactants. Instead, the composition includes an alaninate surfactant, in combination with a specific structuring system combining an emulsifying agent with a rheology modifier as defined herein. Such personal care composition can achieve the desired structure necessary for suspending and stabilizing benefit agents.

The resulting personal care composition exhibits excellent cleansing efficacy, effectively removing dirt, sweat, sebum, and body odors from the skin. Additionally, it ensures the rapid formation of micelles upon dilution, facilitating the deposition of benefit agents onto the skin.

The personal care composition offers an improved rheology or viscosity, providing enhanced texture, spreadability, and foam generation. This leads to a luxurious sensory experience during use, enhancing consumer satisfaction and acceptance of the product.

In conclusion, the present disclosure refers to a sulfate-free personal care composition with an improved rheology, viscosity and lather properties. By utilizing a sulfate-free surfactant system, namely an alaninate surfactant and specific structuring system, the composition achieves the desired balance between structure and micellar formation, lather and resulting in effective skin cleansing and deposition of benefit agents.

### Personal Care Composition

A personal care composition is provided and comprises at least a cleansing phase and a benefit phase.

The personal care composition pertains to a sulfate-substantially free surfactant system. In other words, the personal care composition is substantially free of alkyl sulfate and/or alkyl ether sulfate type of surfactant. Namely, the personal care composition comprises less than about 1.5%, or less than about 1.2%, or less than about 1%, or less than about 0.8%, or less than about 0.5%, or less than about 0.3%, or less than about 0.1%, or less than about 0.01% or is free of alkyl sulfate and/or alkyl ether sulfate type of surfactant by weight of the composition.

Preferably, the personal care composition may comprise less than about 1.5%, or less than about 1.2%, or less than about 1%, or less than about 0.8%, or less than about 0.5%, or less than about 0.3%, or less than about 0.1%, or less than about 0.01%, or is free of any alkyl sulfate which comprises C₁₂-C₁₈ alkyl sulfate and/or any alkyl ether sulfate including alkyl glyceryl ether sulfates.

More preferably, the personal care composition may comprise less than about 1.5%, or less than about 1.2%, or less than about 1%, or less than about 0.8%, or less than about 0.5%, or less than 0.3%, or less than about 0.1%, or less than about 0.01%, or is free of sodium lauryl sulfate.

Alternatively, the personal care composition may be free of alkyl sulfate and/or alkyl ether sulfate type of surfactant. Namely, the personal care composition may comprise 0% of alkyl sulfate and/or alkyl ether sulfate type of surfactant by weight of the composition, thus no detectable amount of alkyl sulfate and/or alkyl ether sulfate type of surfactant.

In that respect, the personal care composition may not comprise any alkyl sulfate which comprises C₁₂-C₁₈ alkyl sulfate and/or any alkyl ether sulfate including alkyl glyceryl ether sulfates.

The personal care composition may not comprise any alkyl ether sulfates which are those having the formula:

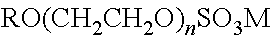

wherein R is an alkyl or alkenyl having 8 to 18 carbons, preferably 12 to 18 carbons, n has an average value of greater than at least 0.5, preferably between 2 and 3; and M is a solubilizing cation such as sodium, potassium, ammonium or substituted ammonium.

The personal care composition may not comprise any ammonium and sodium lauryl ether sulfates.

If the personal care composition does contain alkyl sulfate and/or alkyl ether sulfate type of surfactant, its content of such a weight proportion of alkyl sulfates or alkyl ether sulfate type surfactant may be less than or equal to the sum of 0.6, more preferably less than or equal to the sum of 0.2, even more preferably equal to 0.

The personal care composition may be free of any alkoxylated, preferably ethoxylated anionic surfactant.

The personal care composition may not comprise any structuring anionic surfactant selecting from the group consisting of sodium trideceth (n) sulfate (STnS) wherein n is between 0 and 3, sodium laureth (n) sulfate) wherein n is between 0 and 3, sodium tridecyl sulfate, sodium C₁₂₋₁₃ alkyl sulfate, sodium C₁₂₋₁₅ alkyl sulfate, sodium C₁₁₋₁₅ alkyl sulfate, sodium C₁₂₋₁₈ alkyl sulfate, sodium C₁₀₋₁₆ alkyl sulfate, sodium C₁₂₋₁₃ pareth sulfate, sodium C₁₂₋₁₃ pareth-n sulfate, sodium C₁₂₋₁₄ pareth-n sulfate, and mixtures thereof.

### Cleansing Phase

The personal care composition comprises a cleansing phase. The cleansing phase comprises an alaninate surfactant; a zwitterionic or amphoteric surfactant; and a structuring system.

Optionally, the cleansing phase may comprise an aqueous structured surfactant phase.

### Alaninate Surfactant

The personal care composition or the cleansing phase comprises an alaninate surfactant.

The alaninate surfactant may be selected from the group consisting of sodium cocoyl alaninate, triethylamine cocoyl alaninate, sodium lauroyl alaninate, sodium N-dodecanoyl-1-alaninate, and combinations thereof.

As a preferred aspect, the alaninate surfactant comprises sodium cocoyl alaninate. Sodium cocoyl alaninate is an anionic amino acid from alanine and coconut fatty acid derived surfactant from nature. The alaninate surfactant such as sodium cocoyl alaninate is sulfate free. The material is biodegradable, hypoallergenic, mild to skin and eye. Sodium cocoyl alaninate can help for delivering mild cleansing which imparts pleasant moisturizing feel after drying. Sodium cocoyl alaninate can be sourced from Ajinomoto as Amilite™ ACS-12. Alternatively, sodium cocoyl alaninate can be sourced from Sino-Lion as Eversoft™ ACS-30S having a content of sodium chloride between about 4-6 wt. %, or Eversoft™ ACS having a content of sodium chloride of less than about 1 wt. %, around about 0.2 wt. %.

The personal care composition may comprise from about 5 wt. % to about 20 wt. % of the alaninate surfactant; preferably from about 7 wt. % to about 16 wt. % of the alaninate surfactant; more preferably from about 9 wt. % to about 13 wt. % of the alaninate surfactant; most preferably from about 10 wt. % to about 12 wt. % of the alaninate surfactant.

The personal care composition may comprise from about 5 wt. % to about 20 wt. % of sodium cocoyl alaninate; preferably from about 7 wt. % to about 16 wt. % of sodium cocoyl alaninate; more preferably from about 9 wt. % to about 13 wt. % of sodium cocoyl alaninate; most preferably from about 10 wt. % to about 12 wt. % of sodium cocoyl alaninate.

The personal care composition or the cleansing phase may not comprise any additional anionic surfactants being not an alaninate surfactant.

### Additional Anionic Surfactant

The personal care composition or the cleansing phase may comprise one or more additional anionic surfactants. The additional anionic surfactant is not an alaninate surfactant.

The one or more additional anionic surfactants may be selected from the group consisting of isethionates, sarcosinates, sulfosuccinates, sulfoacetates, acyl glycinates, lactates, lactylates, and mixtures thereof.

The personal care composition or the cleansing phase may comprise from about 0.5% to about 25%, preferably from about 1% to about 10%, more preferably from about 2% to about 5% of the one or more additional anionic surfactants by weight of the composition.

Non-limiting examples of isethionate surfactants can include sodium lauroyl isethionate, sodium lauroyl methyl isethionate, sodium oleoyl isethionate, sodium oleoyl methyl isethionate, sodium stearoyl isethionate, sodium stearoyl methyl isethionate, sodium myristoyl isethionate, sodium myristoyl methyl isethionate, sodium palmitoyl isethionate, sodium palmitoyl methyl isethionate, sodium cocoyl isethionate, sodium cocoyl methyl isethionate, a blend of stearic acid and sodium cocoyl isethionate, ammonium cocoyl isethionate, ammonium cocoyl methyl isethionate, and mixtures thereof.

Non-limiting examples of sarcosinate surfactants can include sodium lauroyl sarcosinate, sodium cocoyl sarcosinate, sodium myristoyl sarcosinate, TEA-cocoyl sarcosinate, ammonium cocoyl sarcosinate, ammonium lauroyl sarcosinate, dimer dilinoleyl bis-lauroyl glutamate/lauroyl sarcosinate, lauroyl sarcosinate, isopropyl lauroyl sarcosinate, potassium cocoyl sarcosinate, potassium lauroyl sarcosinate, sodium oleoyl sarcosinate, sodium palmitoyl sarcosinate, TEA-lauroyl sarcosinate, TEA-oleoyl sarcosinate, TEA-palm kernel sarcosinate, and mixtures thereof.

Non-limiting examples of sulfosuccinate surfactants can include disodium N-octadecyl sulfosuccinate, disodium lauryl sulfosuccinate, diammonium lauryl sulfosuccinate, sodium lauryl sulfosuccinate, disodium laureth sulfosuccinate, tetrasodium N-(1,2-dicarboxyethyl)-N-octadecyl sulfosuccinnate, diamyl ester of sodium sulfosuccinic acid, dihexyl ester of sodium sulfosuccinic acid, dioctyl esters of sodium sulfosuccinic acid, and combinations thereof.

Non-limiting examples of sulfoacetates can include sodium lauryl sulfoacetate, ammonium lauryl sulfoacetate, and combination thereof.

Non-limiting examples of acyl glycinates can include sodium cocoyl glycinate, sodium lauroyl glycinate, and combination thereof.

Non-limiting example of lactates can include sodium lactate.

Non-limiting examples of lactylates can include sodium lauroyl lactylate, sodium cocoyl lactylate, and combination thereof.

In that disclosure, alkyl is defined as a saturated or unsaturated, straight or branched alkyl chain with 6 to 30 carbon atoms, preferably with 8 to 22 carbon atoms, more preferably with 9 to 18 carbon atoms. In that case, acyl is defined as of formula R—C(O)—, wherein R is a saturated or unsaturated, straight or branched alkyl or alkenyl, preferably alkyl chain with 6 to 30 carbon atoms, preferably with 8 to 22 carbon atoms, more preferably with 9 to 18 carbon atoms.

### Zwitterionic or Amphoteric Surfactant

The personal care composition or the cleansing phase comprises a zwitterionic or amphoteric surfactant.

Suitable amphoteric or zwitterionic surfactants can include those described in U.S. Pat. Nos. 5,104,646 and 5,106,609, each of which is incorporated herein by reference.

The personal care composition or the cleansing phase comprises a zwitterionic surfactant.

The personal care composition may comprise from about 0.01 wt. % to about 20 wt. % of the zwitterionic surfactant; preferably from about 0.1 wt. % to about 10 wt. % of the zwitterionic surfactant; more preferably from about 1 wt. % to about 10 wt. % of the zwitterionic surfactant; most preferably from about 2 wt. % to about 5 wt. % of the zwitterionic surfactant.

The zwitterionic surfactant may comprise a betaine. As a preferred aspect, the zwitterionic surfactant may comprise an alkyl betaine or an alkyl amidopropyl betaine.

Examples of betaine zwitterionic surfactants may include coco dimethyl carboxymethyl betaine, cocoamidopropyl betaine (CAPB), coco-betaine, lauryl amidopropyl betaine (LAPB), oleyl betaine, lauryl dimethyl carboxymethyl betaine, lauryl dimethyl alpha-carboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl) alpha-carboxyethyl betaine, and mixtures thereof.

Examples of sulfobetaines may include coco dimethyl sulfopropyl betaine, stearyl dimethyl sulfopropyl betaine, lauryl dimethyl sulfoethyl betaine, lauryl bis-(2-hydroxyethyl) sulfopropyl betaine and mixtures thereof.

The zwitterionic surfactant may be selected from the group consisting of cocamidopropyl betaine, coco-betaine, lauramidopropyl betaine, and mixtures thereof.

Most preferably, the zwitterionic surfactant may comprise cocamidopropyl betaine. In that aspect, the personal care composition may comprise from about 0.01 wt. % to about 20 wt. % of cocamidopropyl betaine; preferably from about 0.1 wt. % to about 10 wt. % of cocamidopropyl betaine; more preferably from about 1 wt. % to about 10 wt. % of cocamidopropyl betaine; most preferably from about 2 wt. % to about 5 wt. % of cocamidopropyl betaine.

Cocamidopropyl betaine can be sourced from BASF as Dehyton® PK 45 having a sodium chloride content between about 5.80-7.30 wt. %. Alternatively, cocamidopropyl betaine can be sourced from Tinci as TC-CAB 35 having salt content below or equal to about 6.0 wt. %; or from Evonik as TEGO BETAIN F-50 having a sodium chloride content between about 5.80-7.30 wt. %; or from Stepan as AMPHOSOL® HCA-HP having a sodium chloride content about 5.2 wt. %. Alternatively, cocamidopropyl betaine can be sourced from Kensing™ as SensaFoam™ CK PH 12/MB having a sodium chloride content of about 5 wt. %.

Alternatively, or in addition to the zwitterionic surfactant, the personal care composition or the cleansing phase may comprise an amphoteric surfactant.

Alternatively, or in addition to the zwitterionic surfactant, the personal care composition may comprise from about 0.01 wt. % to about 20 wt. % of the amphoteric surfactant; preferably from about 0.1 wt. % to about 10 wt. % of the amphoteric surfactant; more preferably from about 1 wt. % to about 10 wt. % of the amphoteric surfactant; most preferably from about 2 wt. % to about 5 wt. % of the amphoteric surfactant.

Additional amphoteric surfactants suitable for use in the cleansing phase can include those surfactants broadly described as derivatives of aliphatic secondary and tertiary amines in which an aliphatic radical can be straight or branched chain and wherein an aliphatic substituent can contain from about 8 to about 18 carbon atoms such that one carbon atom can contain an anionic water solubilizing group, e.g., carboxy, sulfonate, sulfate, phosphate, or phosphonate. Examples of compounds falling within this definition can be 3-(dodecyldimethylammonio)-2-hydroxypropane-1-sulfonate or Lauryl hydroxysultaine, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and products described in U.S. Pat. No. 2,528,378, each of which is incorporated herein by reference. Amphoacetates and diamphoacetates can also be used.

The amphoteric surfactant included in the personal care composition described herein may be preferably selected from the group consisting of sodium lauroamphoacetate, sodium cocoamphoacetate, disodium lauroamphoacetate, disodium cocodiamphoacetate, and mixtures thereof.

Sodium cocoamphoacetate can be sourced from Stepan as AMPHOSOL® 1C having a sodium chloride content between about 6.5 wt. %. Sodium lauroamphoacetate can be sourced from Colonial Chemical, Inc. as Cola®Teric SLAA having a sodium chloride content between about 6.5 wt. % and about 7.5 wt. %

The cleansing phase may further comprise an additional cosurfactant, for example, a nonionic surfactant, Nonionic surfactants suitable for use in the personal care compositions can include those selected from the group consisting of alkyl ethoxylates, alkyl glucosides, polyglucosides (e.g., alkyl polyglucosides, decyl polyglucosides), polyhydroxy fatty acid amides, alkoxylated fatty acid esters, sucrose esters, amine oxides, or mixtures thereof. Some exemplary nonionic surfactants can include cocamide monoethanolamine, decyl glucoside, or a mixture thereof.

### Lamellar Structure

The cleansing phase of the personal care composition may optionally comprise an aqueous structured surfactant phase. The cleansing phase may be comprised of a structured domain that comprises the surfactants as set out hereinabove. The structured domain may be preferably an opaque structured domain, which is preferably a lamellar phase. The lamellar phase produces lamellar vesicles. The lamellar phase can provide resistance to shear, adequate yield to suspend particles and droplets and at the same time provides long term stability, since it is thermodynamically stable.

The personal care composition may be a structured lamellar composition. The personal care composition may comprise at least a 40% lamellar structure, preferably at least a 50% lamellar structure, more preferably at least a 70% lamellar structure.

Alternatively, the personal care composition may comprise a lamellar phase volume from about 40% to about 100%, preferably from about 50% to about 100%, more preferably from about 70% to about 100% of a lamellar phase volume according to the Ultracentrifugation Method disclosed herein.

As an aspect of the disclosure, the personal care composition disclosed herein may also be substantially free of one or more inorganic electrolytes.

Such one or more inorganic electrolytes include halides of alkaline metals, alkaline earth metals, ammonium and other metals, such as aluminum and zinc; sulphates and phosphates of alkaline metals, alkaline earth metals, ammonium and other metals such has aluminum and zinc; and alkaline metal silicates, among others.

As a preferred aspect, the personal care composition may substantially free of one or more inorganic electrolytes including sodium chloride, potassium chloride, sodium sulphate, potassium sulphate, magnesium chloride, magnesium sulphate, magnesium chloride, magnesium sulphate, zinc sulphate, ammonium chloride, and combinations thereof.

As a more preferred aspect, the personal care composition may substantially free of sodium chloride.

The term “substantially free of one or more inorganic electrolytes” as used herein means less than about 1.25%, less than about 1.2%, less than about 1%, less than about 0.8%, less than about 0.5%, less than about 0.3%, less than about 0.1%, less than about 0.01% or less than an immaterial amount of inorganic electrolytes by weight of the composition.

The term “substantially free of sodium chloride” as used herein means less than about 1.25%, less than about 1.2%, less than about 1%, less than about 0.8%, less than about 0.5%, less than about 0.3%, less than about 0.1%, less than about 0.01% or less than an immaterial amount of sodium chloride by weight of the composition.

When having a low level of one or more inorganic electrolytes; or sodium chloride, the % of lamellar structure could be enhanced. Such improvement could help for providing resistance to shear, adequate yield to suspend particles and droplets and at the same time provides long term stability.

To provide a personal care composition being substantially free of one or more inorganic electrolytes; or sodium chloride, the cleansing phase comprises an alaninate surfactant; a zwitterionic or amphoteric surfactant; and a structuring system as set out herein. Also, the personal care composition may not comprise any further inorganic electrolyte or sodium chloride added.

In that case, the alaninate surfactant may have a relatively low content of inorganic electrolytes or sodium chloride. For instance, sodium cocoyl alaninate can be sourced from Sino-Lion as Eversoft™ ACS having a content of sodium chloride of less than about 1 wt. %, around about 0.2 wt. %.

In addition to the alaninate surfactant, the zwitterionic or amphoteric surfactant may have a relatively low content of inorganic electrolytes or sodium chloride. For instance, cocamidopropyl betaine can be sourced from BASF as Dehyton® PK 45 having the sodium chloride content removed, resulting in about 33.05 wt. % dry residue; and about 0.21 wt. % sodium chloride.

### Structuring System

The personal care composition includes a structuring system. The structuring system can help to provide structure to the cleansing phase and stability to the personal care composition. A structuring system includes from about 0.5 wt. % to about 5 wt. % of an emulsifying agent. The emulsifying agent is a glyceryl ester and/or a non-ionic emulsifier having an HLB of from about 3.4 to about 13.0. The structuring system further includes from about 0.01 wt. % to about 10 wt. % of a rheology modifier.

### Emulsifying Agent

The personal care composition comprises a cleansing phase. The cleansing phase comprises a structuring system. The structuring system comprises from about 0.5% to about 5% of an emulsifying agent by weight of the composition.

The personal care composition comprises from about 0.5% to about 5% of an emulsifying agent by weight of the composition.

The personal care composition may comprise from about 1 wt. % to about 3 wt. % of the emulsifying agent; preferably from about 1 wt. % to about 2.75 wt. % of the emulsifying agent; more preferably from about 1 wt. % to about 2.5 wt. % of the emulsifying agent.

The personal care composition may comprise a weight ratio of the alaninate surfactant to the emulsifying agent that is greater than about 5:1 to about 15:1; preferably from about 5.5:1 to about 14:1; more preferably from about 5.6:1 to about 7:1.

With the levels of the emulsifying agent or a weight ratio of the alaninate surfactant to the emulsifying agent as set out hereinbefore, acceptable lather can be obtained.

The emulsifying agent is a glyceryl ester. Alternatively, or in addition, the emulsifying agent is a non-ionic emulsifier having an HLB of from about 3.4 to about 13.0.

The personal care composition may comprise from about 1 wt. % to about 3 wt. % of the glyceryl ester; preferably from about 1 wt. % to about 2.75 wt. % of the glyceryl ester; more preferably from about 1 wt. % to about 2.5 wt. % of the glyceryl ester.

The personal care composition may comprise a weight ratio of the alaninate surfactant to the glyceryl ester that is greater than about 5:1 to about 15:1; preferably from about 5.5:1 to about 14:1; more preferably from about 5.6:1 to about 7:1.

The glyceryl ester is selected from glyceryl laurate, glyceryl caprate, glyceryl caprylate, glyceryl caprylate/caprate, glyceryl stearate, and a mixture thereof.

Other suitable glyceryl esters may be selected such as the glyceryl esters containing C8-C10 mono-di- and tri-glycerides which are different from C8-C10 mono-dicaprylate 1,2,3-propanetriol.

As a preferred aspect, the emulsifying agent comprises glyceryl caprylate/caprate. Glyceryl caprylate/caprate is mild and substantially free of polyethyleneglycol (PEG), Ethylene Oxide/Propylene Oxide (EO/PO), and Nitrogen.

The multifunctional benefits can include yield generation for suspension of particles at relatively high temperature for product stability, viscosity modifier, scalp skin moisturization, wet and dry conditioning, and potential enhanced depo of soluble active. Glyceryl caprylate/caprate can be sourced from Stepan as Stepan-Mild® GCC.

The personal care composition may comprise from about 0.5 wt. % to about 5 wt. % of glyceryl caprylate/caprate; preferably from about 1 wt. % to about 2.75 wt. % of glyceryl caprylate/caprate; more preferably from about 1 wt. % to about 2.5 wt. % glyceryl caprylate/caprate.

As a most preferred aspect, the personal care composition may comprise a weight ratio of sodium cocoyl alaninate to glyceryl caprylate/caprate that is greater than about 5:1 to about 15:1; preferably from about 5.5:1 to about 14:1; more preferably from about 5.6:1 to about 7:1.

Glyceryl caprylate/caprate at the levels set out hereinabove or when combined with sodium cocoyl alaninate at a recited weight ratio can help to improve the lather properties of the composition.

Alternatively, or in addition to a glyceryl ester, preferably glyceryl caprylate/caprate, the emulsifying agent is a non-ionic emulsifier having an HLB of from about 3.4 to about 13.0, preferably about 3.4 to about 8.0.

The personal care composition may comprise from about 1 wt. % to about 3 wt. % of the non-ionic emulsifier having an HLB of from about 3.4 to about 13.0; preferably from about 1 wt. % to about 2.75 wt. % of the non-ionic emulsifier having an HLB of from about 3.4 to about 13.0; more preferably from about 1 wt. % to about 2.5 wt. % of the non-ionic emulsifier having an HLB of from about 3.4 to about 13.0.

The personal care composition may comprise a weight ratio of the alaninate surfactant to the non-ionic emulsifier having an HLB of from about 3.4 to about 13.0 that is greater than about 5:1 to about 15:1; preferably from about 5.5:1 to about 14:1; more preferably from about 5.6:1 to about 7:1.

The balance between the hydrophilic and lipophilic moieties in a surfactant molecule is used as a method of classification (hydrophile-lipophile balance, HLB). The HLB values for commonly-used surfactants are readily available in the literature (e.g., HLB Index in McCutcheon's Emulsifiers and Detergents, MC Publishing Co., 2004). Another way of obtaining HLB values is to estimate by calculations. The HLB system was originally devised by Griffin (J. Soc. Cosmetic Chem., 1, 311, 1949). Griffin defined the HLB value of a surfactant as the mol % of the hydrophilic groups divided by 5, where a completely hydrophilic molecule (with no non-polar groups) had an HLB value of 20. Other examples of how to calculate HLB values are described by Davies in Interfacial Phenomena, 2nd Edition, Academic Press, London, 1963 and by Lin in J. Phys. Chem. 76, 2019-2013, 1972.

The non-ionic emulsifier having an HLB of from about 3.4 to about 13.0 comprises trideceth-3 or trideceth-4.

As a preferred aspect, the emulsifying agent may comprise trideceth-3. In that case, the personal care composition may comprise from about 0.5 wt. % to about 5 wt. % of trideceth-3; preferably from about 1 wt. % to about 2.75 wt. % of trideceth-3; more preferably from about 1 wt. % to about 2.5 wt. % trideceth-3.

The personal care composition may comprise a weight ratio of sodium cocoyl alaninate to trideceth-3 that is greater than about 5:1 to about 15:1; preferably from about 5.5:1 to about 14:1; more preferably from about 5.6:1 to about 7:1.

The non-ionic emulsifier can help to increase the Carreau zero shear viscosity and thus improve the structure and stability of the personal care composition at a specified pH range described more in detailed below.

### Rheology Modifier

The personal care composition comprises from about 0.01 wt. % to about 10 wt. % of the rheology modifier, preferably from about 0.1 wt. % to about 5 wt. % of the rheology modifier, more preferably from about 0.5 wt. % to about 2 wt. % of the rheology modifier, even more preferably from about 0.6 wt. % to about 1.5 wt. %, most preferably from about 1.0 wt. % to about 1.3 wt. % of the rheology modifier.

The rheology modifier may be an associative polymer. Associative polymers are polymers constituted by a hydrophilic main chain and hydrophobic side chains. Their behavior in solution is a result of competition between the hydrophobic and hydrophilic properties of their structure. The hydrophobic units tend to form aggregates constituting linkage points between the macromolecular chains. From a rheological viewpoint, associative water-soluble polymers have a very high viscosifying power in water and retain their viscosity well in a saline medium. In mixed polymer and surfactant systems, surfactant aggregates can form, which are stabilized by diverse types of interactions: electrostatic interactions, dipolar interactions, or hydrogen bonds. Associative water-soluble polymers can interact more specifically with surfactants due to their hydrophobic portions.

The hydrophilic main chain of these associative polymers can, in particular, result from polymerization of a hydrophilic monomer containing functions onto which hydrophobic chains can subsequently be grafted, for example acid functions. This method of preparing associative polymers is described in particular in the “Water Soluble Polymers”, ACS Symposium Series 467, cd. Shalaby W Shalaby et al., Am. Chem. Soc. Washington (1991), pp. 82-200. However, a water-soluble polymer of natural origin, or a natural polymer rendered water-soluble by chemical modification, can also be used. Associative polymers can also be formed by copolymerization of hydrophilic monomers and hydrophobic monomers. These hydrophobic polymers, introduced into the reaction medium in a much smaller quantity than the hydrophilic polymers, generally comprise a fatty hydrocarbon chain. This method of preparation is described in the publication by S. Biggs et. al., J. Phys Chem. (1992, 96. pp 1505-11). Rheology modifiers are substances that are added to the personal care compositions, to modify their flow properties and rheological behavior. Rheology modifiers can alter viscosity, thicken the material, or change its flow characteristics.

The rheology modifier may be selected from the group consisting of a polyacrylate, a polysaccharide, a modified polyol, an hydrophobically modified polyacrylate, an hydrophobically modified polysaccharide, and mixtures thereof.

Specifically, the rheology modifier may be selected from the group consisting of sodium polyacrylate, acrylates copolymer, Acrylates/Vinyl Isodecanoate Crosspolymer, Acrylates/C10-30 Alkyl Acrylate Crosspolymer, Acrylates/C10-30 alkyl acrylate crosspolymer including stearyl side chains with less than about 1% Hydrophobic modification, Acrylates/C10-30 alkyl acrylate crosspolymer including octyl side chains with less than about 5% Hydrophobic modification, Ammonium Acryloyldimethyltaurate/Beheneth-25 Methacrylate Crosspolymer, Acrylates/Beheneth-25 Methacrylate Copolymer, Acrylates/Steareth-20 Methacrylate Copolymer, and Acrylates/Steareth-20 Methacrylate Crosspolymer, PEG-150/Decyl Alcohol/SMDI Copolymer, PEG-150/stearyl alcohol/SMDI copolymer, hydroxypropyl starch phosphate, distarch phosphate, sodium carboxymethyl starch, starch, Tapioca starch, xanthan gum, gellan gum, carboxymethyl cellulose, carboxymethyl hydroxyethyl cellulose, hydroxypropyl methyl cellulose, sodium alginate, and mixtures thereof.

More specifically, the rheology modifier may be selected from the group consisting of sodium polyacrylate, acrylates copolymer, Acrylates/Vinyl Isodecanoate Crosspolymer, Acrylates/C10-30 Alkyl Acrylate Crosspolymer, Ammonium Acryloyldimethyltaurate/Beheneth-25 Methacrylate Crosspolymer, Acrylates/Beheneth-25 Methacrylate Copolymer, Acrylates/Steareth-20 Methacrylate Copolymer, and Acrylates/Steareth-20 Methacrylate Crosspolymer, hydroxypropyl starch phosphate, distarch phosphate, sodium carboxymethyl starch, Tapioca starch, xanthan gum, gellan gum, and mixtures thereof.

Non-limiting examples of associative polymers being a polyacrylate or an hydrophobically modified polyacrylate include sodium polyacrylate, acrylates copolymer, Acrylates/Vinyl Isodecanoate Crosspolymer (Stabylen 30 from 3V), Acrylates/C10-30 Alkyl Acrylate Crosspolymer (Pemulen TR1 and TR2), Aqupec SER-300 made by Sumitomo Seika of Japan, which is Acrylates/C10-30 alkyl acrylate crosspolymer comprising stearyl side chains with less than about 1% HM, Ammonium Acryloyldimethyltaurate/Beheneth-25 Methacrylate Crosspolymer (Aristoflex HMB from Clariant), Acrylates/Beheneth-25 Methacrylate Copolymer (Aculyn 28 from Rohm and Haas); Acrylates/Steareth-20 Methacrylate Copolymer (Aculyn 22 from Rohm and Haas), and Acrylates/Steareth-20 Methacrylate Crosspolymer (Aculyn 88 from Rohm and Haas).

Acrylate copolymers are defined as polymers of two or more monomers consisting of acrylic acid, methacrylic acid (q.v.) or one of their simple esters. Simple esters of methacrylic acid are made with simple alkyl groups such as methyl, ethyl, propyl and butyl and their respective regioisomers. An example of acrylate copolymers may be Luvimer 100 from BASF which is made of a terpolymer of tert-butyl acrylate, ethyl acrylate and methacrylic acid.

Non-limiting examples of associative polymers being a modified polyol include PEG-150/Decyl Alcohol/SMDI Copolymer (Aculyn 44 from Dow Chemical Company), and PEG-150/stearyl alcohol/SMDI copolymer (Aculyn 46 from Dow Chemical Company).

“SMDI” as used herein means saturated methylene diphenyl diisocyanate. “PEG-150/decyl alcohol/SMDI copolymer” is a copolymer of PEG-150 (q.v.), Decyl Alcohol (q.v.), and Saturated Methylene Diphenyl Diisocyanate (q.v.) (SMDI) monomers. “PEG-150/stearyl alcohol/SMDI copolymer” is a copolymer of PEG-150 (q.v.), Saturated Methylene Diphenyl Diisocyanate (q.v.) (SMDI), and Stearyl Alcohol (q.v.) monomers.

Preferably, the rheology modifier may comprise acrylates/C10-30 alkyl acrylate crosspolymer. Acrylates/C10-30 alkyl acrylate Crosspolymer is a copolymer of C10-30 alkyl acrylate and one or more monomers of acrylic acid, methacrylic acid or one of their simple esters crosslinked with an allyl ether of sucrose or an allyl ether of pentaerythritol.

An exemplary preferred acrylates/C10-30 alkyl acrylate crosspolymer may be Aqupec SER-300 made by Sumitomo Seika of Japan, which is Acrylates/C10-30 alkyl acrylate crosspolymer comprising stearyl side chains with less than about 1% Hydrophobic modification (HM). Other preferred rheology modifiers in that category may comprise stearyl, octyl, decyl and lauryl side chains.

Preferred acrylates/C10-30 alkyl acrylate crosspolymer may be Aqupec SER-150 that is acrylates/C10-30 alkyl acrylates crosspolymer comprising about C18 (stearyl) side chains and about 0.4% HM, and Aqupec HV-701EDR that is acrylates/C10-30 alkyl acrylates crosspolymer which comprises about C8 (octyl) side chains and about 3.5% HM.

The crosslinked rheology modifier may include a percentage hydrophobic modification, which is the mole percentage of monomers expressed as a percentage of the total number of all monomers in the polymer backbone, including both acidic and other non-acidic monomers. The percentage hydrophobic modification of the polymer, hereafter % HM, can be determined by the ratio of monomers added during synthesis, or by analytical techniques such as proton nuclear magnetic resonance (NMR). The alkyl side chain length can be determined similarly.

The structuring system of the cleansing phase comprises from about 0.01% to about 5%, preferably from about 0.01% to about 1%, more preferably from about 0.02% to about 0.3%, most preferably from about 0.03% to about 0.1% by weight of the personal care composition, of acrylates/C10-30 alkyl acrylate crosspolymer.

Non-limiting example of an associative polymer being a polysaccharide, or a modified polysaccharide includes starch, Tapioca starch, xanthan gum, gellan gum, carboxymethyl cellulose, carboxymethyl hydroxyethyl cellulose, hydroxypropyl methyl cellulose, sodium alginate, and mixtures thereof.

The rheology modifier may comprise xanthan gum. Xanthan gum can help to improve the stability of the personal care composition.

The structuring system of the cleansing phase may comprise from about 0.01% to about 10%, preferably from about 0.1% to about 5%, more preferably from about 0.3% to about 2%, most preferably from about 0.5% to about 1% by weight of the personal care composition, of xanthan gum.

Alternatively, the rheology modifier may comprise a hydrophobically modified polysaccharide, especially a modified starch. The modified starch may be selected from the group consisting of hydroxypropyl starch phosphate, distarch phosphate, sodium carboxymethyl starch, and mixtures thereof.

In particular, the modified starch may comprise hydroxypropyl starch phosphate. Hydroxypropyl starch phosphate may be provided as Structure® XL from Nouryon, or C*HiForm™ A12747 from Cargill. Distarch phosphate may be provided as Agenajel 20.306 from Agrana Stärke. Sodium carboxymethyl starch may be provided as Vivastar® CS Instant Powder from J. Rettenmaier & Söhne.

Starch is a carbohydrate polymer consisting of a large number of glucose units linked together primarily by alpha 1-4 glucosidic bonds. The starch polymers come in two forms: linear (amylose) and branched through alpha 1-6 glucosidic bonds (amylopectin), with each glucose unit possessing a maximum of three hydroxyls that can undergo chemical substitution.

Hydroxypropyl starch phosphate is a modified starch. It is obtained in accordance with good manufacturing practice by esterification of food starch with sodium trimetaphosphate or phosphorus oxychloride combined with etherification by propylene oxide. Hydroxypropylation results in substitution of hydroxyl groups with 2-hydroxypropyl ether. In cases of cross-linking, where phosphorus oxychloride, connects two chains, the structure can be represented by: Starch-O—R—O-Starch, where R=cross-linking group and Starch refers to the linear and/or branched structure.

As a preferred aspect, the rheology modifier may comprise an hydrophobically modified polysaccharide being a modified starch. The modified starch may comprise hydroxypropyl starch phosphate.

In that aspect, the personal care composition may comprise from about 0.01 wt. % to about 10 wt. % of hydroxypropyl starch phosphate, preferably from about 0.1 wt. % to about 5 wt. % of hydroxypropyl starch phosphate, more preferably from about 0.5 wt. % to about 1.5 wt. % of hydroxypropyl starch phosphate, most preferably from about 0.6 wt. % to about 1.0 wt. % of hydroxypropyl starch phosphate.

Such rheology modifiers can help to provide significant enhancement of structure to the cleansing phase and thus the personal care composition, especially when the personal care composition comprises reduced levels of emulsifying agents; and provide said structure at relatively low levels of rheology modifiers. Also, lather can be further improved.

Another aspect may be related to the combination of two rheology modifiers. In that respect, the personal care composition may comprise a mixture of hydroxypropyl starch phosphate and xanthan gum.

The personal care composition may comprise from about 0.3 wt. % to about 1.5 wt. % of hydroxypropyl starch phosphate and from about 0.1 wt. % to about 0.5 wt. % of xanthan gum, preferably from about 0.3 wt. % to about 1.0 wt. % of hydroxypropyl starch phosphate and from about 0.1 wt. % to about 0.4 wt. % of xanthan gum.

The composition can achieve the desired balance between improved structure and improved lather and resulting in effective skin cleansing and deposition of benefit agents.

The personal care composition may comprise a lather volume from about 375 mL to about 575 mL, preferably from about 395 mL to about 560 mL, more preferably from about 400 mL to about 545 mL, most preferably from about 450 mL to about 540 mL as measured according to the Cylinder Method as disclosed herein.

### Deposition Polymer

The personal care composition may additionally comprise a cationic deposition polymer in the cleansing phase as a deposition aid for the benefit agents described herein.

Suitable cationic deposition polymers for use in the compositions may contain cationic nitrogen-containing moieties such as quaternary ammonium moieties. Non-limiting examples of cationic deposition polymers for use in the personal care composition include cationic cellulose derivatives. Preferred cationic cellulose polymers are the salts of hydroxyethyl cellulose reacted with trimethyl ammonium substituted epoxide, referred to in the industry (CTFA) as Polyquaternium 10 which are available from Amerchol Corp. (Edison, N.J., USA) in their Polymer KG, JR and LR series of polymers with the most preferred being KG-30M. Other suitable cationic deposition polymers include cationic guar gum derivatives, such as guar hydroxypropyltrimonium chloride, specific examples of which include the Jaguar series (preferably Jaguar C-17) commercially available from Rhodia Inc., and N-Hance polymer series commercially available from Aqualon.

The cationic deposition polymers of the personal care composition may have a cationic charge density from about 0.8 meq/g to about 2.0 meq/g, alternatively from about 1.0 meq/g to about 1.5 meq/g.

The personal care composition may comprise from about 0.01% to about 5%, preferably from about 0.1% to about 2%, more preferably from about 0.2% to about 1%, most preferably from about 0.3% to about 1% by weight of the personal care composition, of a cationic deposition polymer.

In an aspect, the personal care composition may comprise from about 0.01% to about 5%, preferably from about 0.1% to about 2%, more preferably from about 0.2% to about 1%, most preferably from about 0.3% to about 1% by weight of the personal care composition, of guar hydroxypropyltrimonium chloride.

### Water

The cleansing phase of the personal care composition may comprise water. The cleansing phase of the personal care composition may comprise from about 10% to about 90%, alternatively from about 40% to about 85%, alternatively from about 60% to about 80% by weight of water.

pH

The pH of the personal care composition is from about 4.0 to about 5.5, preferably from about 4.2 to about 5.3, more preferably from about 4.5 to about 5.25, most preferably from about 4.90 to about 5.25. The pH of the personal care composition can help to provide a structured cleansing phase.

A variety of compounds may be used to adjust the pH value of a composition. Such suitable compounds can include, but are not limited to, citric acid, acetic acid, hydrochloric acid, triethylamine, diethylamine, ethylamine, monoethanol amine, diethanol amine, triethanol amine and any mixtures thereof. The personal care composition may comprise greater than about 0% to about 3% of the pH adjusting agent by weight of the composition, preferably wherein the pH adjusting agent comprises citric acid.

Set up the pH of the personal care composition as recited herein can help to prevent phase separation of the personal care composition. Then, the surfactant levels and/or can be optimized as described herein for building and improving the rheology or viscosity profile of the personal care composition.

### Preservative

The personal care composition may comprise from about 0.01% to about 1.0%, preferably from about 0.02% to about 0.4%, more preferably from about 0.05% to about 0.2%, most preferably from about 0.05% to about 0.1% of a preservative by weight of the composition.

The preservative may include a salicylate salt and a benzoate salt, wherein a total amount of the salicylate salt and the benzoate salt is from about 0.2% to about 1.0%, preferably from about 0.5% to about 0.90%, more preferably from about 0.75% to about 0.85%, by weight of the composition.

The weight ratio of the salicylate salt to the benzoate salt may be from about 1:1.10 to about 1:1.20, preferably from about 1:1.125 to about 1:1.175.

The salicylate salt may be sodium salicylate. The benzoate salt may be sodium benzoate.

### Benefit Phase

The personal care composition comprises a benefit phase. The benefit phase in the personal care composition may be hydrophobic or essentially anhydrous and may be substantially free of water. The benefit phase may be substantially free or free of surfactant. As a preferred aspect, the benefit phase may be anhydrous.

The benefit phase typically comprises a benefit agent. A benefit agent may include water-insoluble or hydrophobic benefit agent. The benefit phase comprises from about 0.1% to about 50%; preferably from about 1% to about 30%; more preferably from about 5% to about 30%, by weight of the personal care composition, of a benefit agent.

Alternatively, the personal care composition comprises from about 0.1 wt. % to about 50 wt. % of the benefit agent; preferably from about 0.5 wt. % to about 15 wt. % of the benefit agent; more preferably from about 1 wt. % to about 10 wt. % of the benefit agent; most preferably from about 2 wt. % to about 10 wt. % of the benefit agent.

The personal care composition may comprise a Carreau Zero Shear Viscosity from about 200 Pa·s to about 16 000 Pa·s, preferably from about 500 Pa·s to about 13 000 Pa·s, more preferably from about 1000 Pa·s to about 12000 Pa·s, even more preferably from about 2900 Pa·s to about 11775 Pa·s, most preferably from about 4500 Pa·s to about 11660 Pa·s, or from about 500 Pa·s to about 7750 Pa·s as measured according to the Carreau Zero Shear Viscosity Method as disclosed herein.

Alternatively, the personal care composition comprises from about 0.1 wt. % to about 50 wt. % of the benefit agent; preferably from about 0.5 wt. % to about 15 wt. % of the benefit agent; more preferably from about 10 wt. % to about 15 wt. % of the benefit agent.

The personal care composition may comprise a Carreau Zero Shear Viscosity from about 200 Pa·s to about 16 000 Pa·s, preferably from about 500 Pa·s to about 13 000 Pa·s, more preferably from about 1000 Pa·s to about 12000 Pa·s, even more preferably from about 2900 Pa·s to about 11775 Pa·s, most preferably from about 4500 Pa·s to about 11660 Pa·s, or from about 1 500 Pa·s to about 16 000 Pa·s as measured according to the Carreau Zero Shear Viscosity Method as disclosed herein.

The hydrophobic skin benefit agent for use in the benefit phase of the composition may have a Vaughan Solubility Parameter (VSP) of from about 5 to about 15, preferably from about 5 to less than 10. These solubility parameters are well known in the formulation arts, and are defined by Vaughan in Cosmetics and Toiletries, Vol. 103, p 47-69, October 1988.

The benefit agent may be selected from the group consisting of petrolatum; lanolin; derivatives of lanolin; natural waxes; synthetic waxes; volatile organosiloxanes; derivatives of volatile organosiloxanes; non-volatile organosiloxanes; derivatives of non-volatile organosiloxanes; lanolin oil; lanolin esters; natural triglycerides; synthetic triglycerides; and mixtures thereof.

Alternatively, non-limiting examples glycerides suitable for use as hydrophobic skin benefit agents herein include castor oil, soybean oil, derivatized soybean oils such as maleated soybean oil, safflower oil, cotton seed oil, corn oil, walnut oil, peanut oil, olive oil, cod liver oil, almond oil, avocado oil, palm oil and sesame oil, vegetable oils, sunflower seed oil, and vegetable oil derivatives; coconut oil and derivatized coconut oil, cottonseed oil and derivatized cottonseed oil, jojoba oil, cocoa butter, shea butter, and mixtures thereof.

Non-limiting examples of acetoglyceride esters suitable for use as hydrophobic skin benefit agents herein include acetylated monoglycerides.

Non-limiting examples of alkyl esters suitable for use as hydrophobic skin benefit agents herein include isopropyl esters of fatty acids and long chain esters of long chain (i.e. C10-C24) fatty acids, e.g. cetyl ricinoleate, non-limiting examples of which include isopropyl palmitate, isopropyl myristate, cetyl riconoleate and stearyl riconoleate. Other examples are: hexyl laurate, isohexyl laurate, myristyl myristate, isohexyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, acyl isononanoate lauryl lactate, myristyl lactate, cetyl lactate, and mixtures thereof.

Non-limiting examples of alkenyl esters suitable for use as hydrophobic skin benefit agents herein include oleyl myristate, oleyl stearate, oleyl oleate, and mixtures thereof.

Non-limiting examples of polyglycerin fatty acid esters suitable for use as hydrophobic skin benefit agents herein include decaglyceryl distearate, decaglyceryl diisostearate, decaglyceryl monomyristate, decaglyceryl monolaurate, hexaglyceryl monooleate, and mixtures thereof.

Non-limiting examples of lanolin and lanolin derivatives suitable for use as hydrophobic skin benefit agents herein include lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate, and mixtures thereof.

Non-limiting examples of silicone oils suitable for use as hydrophobic skin benefit agents herein include dimethicone copolyol, dimethylpolysiloxane, diethylpolysiloxane, mixed C1-C30 alkyl polysiloxanes, phenyl dimethicone, dimethiconol, and mixtures thereof. Preferred are non-volatile silicones selected from dimethicone, dimethiconol, mixed C1-C30 alkyl polysiloxane, and mixtures thereof. Nonlimiting examples of silicone oils useful herein are described in U.S. Pat. No. 5,011,681 (Ciotti et al.).

Still other suitable hydrophobic skin benefit agents include milk triglycerides (e.g., hydroxylated milk glyceride) and polyol fatty acid polyesters.

Still other suitable hydrophobic skin benefit agents include wax esters, non-limiting examples of which include beeswax and beeswax derivatives, spermaceti, myristyl myristate, stearyl stearate, and mixtures thereof. Also useful are vegetable waxes such as carnauba and candelilla waxes; sterols such as cholesterol, cholesterol fatty acid esters; and phospholipids such as lecithin and derivatives, sphingo lipids, ceramides, glycosphingo lipids, and mixtures thereof.

The benefit agent may be selected from argan oil, castor oil, soybean oil, derivatized soybean oils, maleated soybean oil, safflower oil, cotton seed oil, corn oil, walnut oil, peanut oil, olive oil, cod liver oil, sweet almond oil, almond oil, avocado oil, palm oil and sesame oil, vegetable oils, sunflower seed oil, and vegetable oil derivatives; coconut oil and derivatized coconut oil, cottonseed oil and derivatized cottonseed oil, jojoba oil, cocoa butter, shea butter, groundnut oil, camellia oil, beauty-leaf oil, rapeseed oil, coconut kernel, coriander oil, marrow oil, wheat germ oil, jojoba oil or liquid jojoba wax, linseed oil, macadamia oil, corn germ oil, hazelnut oil, walnut oil, vemonia oil, apricot kernel oil, olive oil, evening-primrose oil, palm oil, passion flower oil, grapeseed oil, rose oil, castor oil, rye oil, sesame oil, rice bran oil, camelina oil, soybean oil, sunflower oil, pracaxi oil, babassu oil, mongongo oil, marula oil, arara oil, shea butter oil, Brazil nut oil; or alternatively caprylic/capric acid triglycerides and mixtures thereof.

In a preferred aspect, the benefit agent may be selected from argan oil, castor oil, soybean oil, maleated soybean oil, avocado oil, coconut oil, jojoba oil, cocoa butter, shea butter, and mixtures thereof.

In a more preferred aspect, the benefit agent may be selected from argan oil, soybean oil, maleated soybean oil, shea butter, and mixtures thereof.

In a most preferred aspect, the benefit agent may comprise soybean oil or shea butter. In that aspect, the personal care composition may comprise from about 0.5 wt. % to about 15 wt. % of soybean oil or shea butter; preferably from about 1 wt. % to about 10 wt. % of soybean oil or shea butter, most preferably from about 2 wt. % to about 10 wt. % of soybean oil or shea butter.

The benefit phase may comprise a hydrophobic benefit agent and optionally a lipid bilayer structurant. The lipid bilayer structurant may comprise glyceryl monooleate, glyceryl monostearate, glyceryl monolaurate, or a mixture thereof. The benefit agent may comprise argan oil, soybean oil, maleated soybean oil, shea butter, or a mixture thereof.

### Physical Contact Between the Cleansing and Benefit Phases

In the personal care composition, the cleansing phase and the benefit phase may be in physical contact. The phases may be blended or mixed to a significant degree, but still be physically distinct such that the physical distinctiveness is undetectable to the naked eye.

The phases can also be made to occupy separate and distinct physical spaces inside a package in which the phases can be stored. In such an arrangement, the structured cleansing phase and the benefit phase can be stored such that the phases are not in direct contact with one another.

Alternatively, the personal care composition may be a multiphase personal care composition. In that aspect, the phases of the personal care composition may be made to occupy separate but distinct physical spaces inside the package in which they are stored, but are in direct contact with one another (i.e., they are not separated by a barrier and they are not emulsified or mixed to any significant degree).

The cleaning phase and the benefit phase can be in physical contact while remaining visibly distinct to give, for example, a striped or marbled or geometric configuration.

### Optional Ingredients

As can be appreciated, the compositions described herein may include a variety of optional components to tailor the properties and characteristics of the composition. As can be appreciated, suitable optional components are well known and can generally include any components which are physically and chemically compatible with the essential components of the compositions described herein. Optional components should not otherwise unduly impair product stability, aesthetics, or performance. Individual concentrations of optional components can generally range from about 0.001% to about 10%, by weight of the composition. Optional components can be further limited to components which will not impair the clarity of a translucent composition.

Still, the personal care composition may not include or may be free of direct dyes, oxidative dyes, parabens, or mixtures thereof.

Optional components may include, but are not limited to perfume, dyes, pigments, humectants, conditioning agents, skin exfoliating agents, anti-dandruff actives, and chelating agents. Additional suitable optional ingredients include but are not limited to particles, anti-microbials, foam boosters, anti-static agents, moisturizing agents, propellants, self-foaming agents, pearlescent agents, opacifiers, sensates, suspending agents, solvents, diluents, anti-oxidants, vitamins, and mixtures thereof.

The personal care composition may further comprise from about 0.01% to about 2% of a perfume by weight of the composition, preferably from about 0.1% to about 1.75% of a perfume by weight of the composition, more preferably from about 0.5% to about 1.6% of a perfume by weight of the composition, even more preferably from about 0.8% to about 1.5% of a perfume by weight of the composition.

Typically, the perfume may be a blend of perfumes and aroma chemicals. As used herein, “fragrance” is used to indicate any odoriferous material.

A wide variety of chemicals are known as fragrances in the perfume, including alcohols, aldehydes, ketones, and esters. Non-limiting examples of the fragrances useful herein include pro-fragrances such as acetal pro-fragrances, ketal pro-fragrances, ester pro-fragrances, hydrolyzable inorganic-organic pro-fragrances, and mixtures thereof. The fragrances may be released from the pro-fragrances in a number of ways. For example, the fragrance may be released as a result of simple hydrolysis, or by a shift in an equilibrium reaction, or by a pH-change, or by enzymatic release. The fragrances herein may be relatively simple in their chemical make-up, comprising a single chemical, or may comprise highly sophisticated complex mixtures of natural and synthetic chemical components, all chosen to provide any desired odor.

Suitable fragrances are also disclosed in U.S. Pat. Nos. 4,145,184, 4,209,417, 4,515,705, and 4,152,272, each of which is incorporated herein by reference. Non-limiting examples of fragrances include animal fragrances such as musk oil, civet, castoreurn, ambergris, plant fragrances such as nutmeg extract, cardomon extract, ginger extract, cinnamon extract, patchouli oil, geranium oil, orange oil, mandarin oil, orange Hower extract, cedarwood, vetyver, lavandin, ylang extract, tuberose extract, sandalwood oil, bergamot oil, rosemary oil, spearmint oil, peppermint oil, lemon oil, lavender oil, citronella oil, chamomille oil, clove oil, sage oil, neroli oil, labdanum oil, eucalyptus oil, verbena oil, mimosa extract, narcissus extract.

Other examples of suitable fragrances include, but are not limited to, chemical substances such as acetophenone, adoxal, aldehyde C-12, aldehyde C-14, aldehyde C-18, allyl caprylate, ambroxan, amyl acetate, dimethylindane derivatives, «-amylcinnamic aldehyde, anethole, anisaldehyde, benzaldehyde, borneol, butyl acetate, camphor, carbitol, cinnamaldehyde, cinnamyl acetate, cinnamyl alcohol, cis-3-hexanol and ester derivatives, cis-3-bexenyl methyl carbonate, citral, citronnellol and ester derivatives, cumin aldehyde, cyclamen aldehyde, cyclogalbanate, damascones, decalactone, decanol, estragole, dihydromyrcenol, dimethyl benzyl carbinol, 6,8-dimethyl-2-nonanol, dimethyl benzyl carbinyl butyrate, ethyl acetate, ethyl isobutyrate, ethyl butyrate, ethyl propionate, ethyl caprylate, ethyl cinnamate, ethyl hexanoate, ethyl valerate, ethyl vanillin, eugenol, exaltoiide, fenchone, fruity esters such as ethyl 2-methyl butyrate, galaxolide, geraniol and ester derivatives, helional, 2-heptonone, hexenol, α-hexylcinnamic aldehyde, hydroxycitronellal, indole, isoamyl acetate, isoeugenol acetate, ionones, isoeugenol, isoamyl iso-valerate, iso E super, limonene, linalool, lilial, linalyl acetate, lyral, majantol, mayol, melonal, menthol, p-methylacetophenone, methyl anthranilate, methyl cedrylone, methyl dibydrojasmonate, methyl eugenol, methyl ionone, methyl-α-naphthyl ketone, methylphenylcarbinyl acetate, mugetanol, γ-nonalactone, octanal, phenyl ethyl acetate, phenylacetaldehyde dimethyl acetate, phenoxyethyl isobutyrate, phenyl ethyl alcohol, pinenes, sandalore, santaiol, stemone, thymol, terpenes, triplal, triethyl citrate, 3,3,5-trimethylcyclohexanol, γ-undecalactone, undecenal, vanillin, veloutone, verdox, and mixtures thereof.

### Method

A method of making a liquid personal care composition having an acceptable lather or providing a substantial amount of foam or lather when agitated or mixed with water is provided and comprises the step of forming a personal care composition as set out hereinbefore.

The step of forming the personal care composition may comprise the following steps, preferably in that order:
Adding water;
Forming a cleansing phase by adding a zwitterionic or amphoteric surfactant, optionally following by a preservative as disclosed herein;
Adding a structuring system comprising from about 0.01% to about 10% of a rheology modifier by weight of the composition to obtain a first mixture;
Adding an alaninate surfactant followed by from about 0.5% to about 5% of an emulsifying agent by weight of the composition; wherein the emulsifying agent is a glyceryl ester and/or a non-ionic emulsifier having an HLB of from 3.4 to 13.0; wherein the glyceryl ester is selected from glyceryl laurate, glyceryl caprate, glyceryl caprylate, glyceryl caprylate/caprate, glyceryl stearate, and a mixture thereof; wherein the non-ionic emulsifier having an HLB of from 3.4 to 13.0 comprises trideceth-3 or trideceth-4 to form a cleansing phase;
Optionally continuously mixing until obtaining a homogenous mixture;
Adjusting the pH, preferably by adding citric acid, such that the personal care composition has a pH that is from about 4.0 to about 5.5, preferably from about 4.2 to about 5.3, more preferably from about 4.5 to about 5.25, most preferably from about 4.90 to about 5.25;
Optionally adding a perfume;
Adding from about 0.1% to about 50% of a benefit agent by weight of the composition to obtain a personal care composition comprising at least the cleansing phase and a benefit phase, wherein the composition is substantially free of alkyl sulfate and alkyl ether sulfate type of surfactants.

The emulsifying agent being a glyceryl ester may be preheated before being added to the first mixture and before pH adjustment. Typically, the batches including glyceryl caprylate/caprate or glyceryl laurate may be heated to 60-65° C. then cooled to 30-35° C. before pH adjustment.

The benefit agent may be mixed to the cleansing phase through a Hauschild SpeedMixer™ (Model DAC, 400FV available from FleckTeck, Inc USA) at 1950 rpm for 60 seconds.

Alternatively, the step of forming the personal care composition may comprise the following steps, preferably in that order:
Adding water;
Forming a cleansing phase by adding a zwitterionic or amphoteric surfactant, optionally following by a preservative as disclosed herein;
Adding a predispersion of from about 0.01% to about 10% of a rheology modifier by weight of the composition and from about 0.5% to about 5% of an emulsifying agent, wherein the emulsifying agent is a glyceryl ester and/or a non-ionic emulsifier having an HLB of from 3.4 to 13.0; wherein the glyceryl ester is selected from glyceryl laurate, glyceryl caprate, glyceryl caprylate, glyceryl caprylate/caprate, glyceryl stearate, and a mixture thereof; wherein the non-ionic emulsifier having an HLB of from 3.4 to 13.0 comprises trideceth-3 or trideceth-4;
Adding an alaninate surfactant followed by from about 0.5% to about 5% of an emulsifying agent by weight of the composition to form a cleansing phase;
Optionally continuously mixing until obtaining a homogenous mixture;
Adjusting the pH, preferably by adding citric acid, such that the personal care composition has a pH that is from about 4.0 to about 5.5, preferably from about 4.2 to about 5.3, more preferably from about 4.5 to about 5.25, most preferably from about 4.90 to about 5.25;
Optionally adding a perfume;
Adding from about 0.1% to about 50% of a benefit agent by weight of the composition to obtain a personal care composition comprising at least the cleansing phase and a benefit phase, wherein the composition is substantially free of alkyl sulfate and alkyl ether sulfate type of surfactants.

Typically, the batches including glyceryl caprylate/caprate or glyceryl laurate may be heated to 60-65° C. then cooled to 30-35° C. before pH adjustment.

The benefit agent may be mixed to the cleansing phase through a Hauschild SpeedMixer™ (Model DAC, 400FV available from FleckTeck, Inc USA) at 1950 rpm for 60 seconds.

### Forms and Uses

### Product Form

The personal care composition may be presented in typical personal care formulations. They may be in the form of solutions, dispersion, emulsions, foams, and other delivery mechanisms. The personal care composition may be a rinse-off composition.

The personal care composition may be extrudable or dispensable from a single chamber package. The personal care compositions can be in the form of liquid, semi-liquid, cream, lotion or gel, or solid compositions intended for topical application to skin.

Examples of personal care compositions, preferably personal cleansing compositions can include but are not limited to body wash, moisturizing body wash, foaming body wash, shower gels, a shower or bath cream, skin cleansers, cleansing milks, body wash, in shower body moisturizer, gel, emulsion, oil, mousse or spray.

The personal care composition may not be in the form of a liquid hand wash or a liquid hand sanitizer.

The product forms contemplated for purposes of defining the personal care compositions and methods are rinse-off formulations by which it is meant that the product is applied topically to the skin and then subsequently (i.e., within minutes) rinsed away with water, or otherwise wiped off using a substrate or other suitable removal means.

### Uses

The personal care composition as set out hereinabove may be used for improving the lather of the composition.

The personal care composition as set out hereinabove may be used for suspending benefits agents selected from the group consisting of hair care and skin care benefit agents, particulates, particles, preferably silica and titanium oxide, microcapsules, oils, droplets, pigments, opacifiers, pearlescent agents, feel modifiers, oil absorbers, skin protectants, matting agents, friction enhancers, slip agents, conditioning agents, exfoliants, odor absorbers, or cleaning enhancers, and mixtures thereof.

The personal care composition can advantageously provide relatively improved ecotoxic or ecologically friendly environmental profile.

The personal care composition can help to provide good aesthetic properties such as good foam, and is thick and creamy in texture, is silky to the touch and affords conditioning.

### Test Methods

It is understood that the test methods that are disclosed in the Test Methods Section of the present application should be used to determine the respective values of the parameters of the personal care compositions described and claimed herein.

### Cylinder Method

Lather can be measured in accordance with the Cylinder Method. Lather volume is measured using a graduated cylinder and a rotating mechanical apparatus. A 1,000 ml graduated cylinder is used which is marked in 10 ml increments, has a height of 14.5 inches at the 1,000 ml mark from the inside of its base, and has a neck at its top fitted for a plastic insert cap (for example, Pyrex No. 2982). Moderately hard water (about 7 gpg or about 120 ppm) is prepared by dissolving 1.14 grams calcium chloride dihydrate and 1.73 grams magnesium chloride hexahydrate into one U.S. gallon distilled water. The water is maintained at between 40.5-43.3° C. (105-110° F.). The graduated cylinder is heated to about the same temperature by flushing with excess tap water at the same temperature for about 15 seconds, then drying its exterior and shaking briefly upside down to dry the interior. 100.0 grams of the moderately hard water at the indicated temperature is weighed directly into the graduated cylinder. The cylinder is clamped in a mechanical rotating device, which clamps the cylinder vertically with an axis of rotation that transects the center of the graduated cylinder. Using a 3- or 4-place metric balance, invert the plastic cap for the graduated cylinder onto the balance pan and weigh 0.500 grams of composition for compositions less than 19% surfactant (weigh 0.250 grams of composition for compositions greater or equal than 19% surfactant) to within 4 milligrams accuracy, using a holder to keep the cap level. Insert the cap into the graduated cylinder neck while being careful that all composition is now in the space in the cylinder interior. For compositions with very low viscosity which will not remain on the cap surface, 500 mg composition can be added directly to the graduated cylinder. Rotate the cylinder for 25 complete revolutions at a rate of about 10 revolutions per 18 seconds to create a lather and stop in a level, vertical position. When the cylinder stops in a vertical position, start a digital stopwatch. Observing the water draining at the bottom, record the time to the nearest second when the water height measures 50 cc, then 60 cc, then 70 cc and so on until at least 90 cc has drained. Measure and record the total height of the foam in the column interior, which is the lather volume. If the top surface of the lather is uneven, the lowest height at which it is possible to see halfway across the graduated cylinder is the lather volume (ml). If the lather is coarse such that a single or only a few foam cells (“bubbles”) reach across the entire cylinder, the height at which at least about 10 foam cells are required to fill the space is the lather volume, also in ml up from the base. When measuring the lather height, bubbles that are larger than about 25.4 mm (1 inch) across at the top surface are considered free air and not lather. The measurement is repeated and at least three results averaged to obtain the lather volume. In a spreadsheet, calculate the lather density at each observed time point as the volume of foam (total height minus water height) divided by the weight of the foam (100.5 grams minus the weight of water observed, using a density of 1.00 g/cc for water). Fit the 3 time points closest to (ideally, also bracketing) 20 seconds to a 2nd order polynomial equation. Solve the equation for the lather density at 20 seconds, which is the lather density of the composition. Multiply the lather volume by the lather density to obtain the lather mass, in grams.

The entire measurement process should take less than about 3 minutes in order to maintain the desired temperature.

The personal care composition may comprise a lather volume from about 375 mL to about 575 mL, preferably from about 395 mL to about 560 mL, more preferably from about 400 mL to about 545 mL, most preferably from about 450 mL to about 540 mL as measured according to the Cylinder Method as disclosed herein.

### Carreau Zero Shear Viscosity Method

The Carreau Zero Shear Viscosity of a material which is a phase or a composition of the personal care composition, can be measured either prior to combining in the composition, after preparing a composition, or first separating a phase or component from a composition by suitable physical separation means, such as centrifugation, pipetting, cutting away mechanically, rinsing, filtering, or other separation means.

A controlled stress rheometer such as a TA Instruments Discovery HR2 Rheometer is used to determine the Carreau Zero Shear Viscosity. The determination is performed at 25° C. with the 4 cm diameter parallel plate measuring system and a 1 mm gap. The geometry has a shear stress factor of 79580 m⁻³ to convert torque obtained to stress. Serrated plates can be used to obtain consistent results when slip occurs.

First the material (i.e. the sample to be tested) is positioned on the rheometer base plate, the measurement geometry (upper plate) is moved into position 1.1 mm above the base plate. Excess material at the geometry edge is removed by scraping after locking the geometry. The geometry is then moved to the target 1 mm position above the base plate and a pause of about 1 minute is allowed to allow loading stresses to relax. This loading procedure ensures no tangential stresses are loaded at the measurement onset, which can influence results obtained. If the material comprises particles discernible to the eye or by feel (beads, e.g.) which are larger than about 150 microns in number average diameter, the gap setting between the base plate and upper plate is increased to the smaller of 4 mm or 8-fold the diameter of the 95th volume percentile particle diameter. If a phase has any particle larger than 5 mm in any dimension, the particles are removed prior to the measurement.

The measurement is performed by applying a continuous shear stress ramp from 0.1 Pa to 1,000 Pa over a time interval of 4 minutes using a logarithmic progression, i.e., measurement points evenly spaced on a logarithmic scale. Thirty (30) measurement points per decade of stress increase are obtained. If the measurement result is incomplete, for example if material is observed to flow from the gap, results obtained are evaluated with incomplete data points excluded. If there are insufficient points to obtain an accurate measurement, the measurement is repeated with an increased number of sample points.

The Carreau Zero Shear Viscosity (Pa·s) is obtained by fitting the data to a Carreau viscosity model.

The personal care composition may have a Carreau Zero Shear Viscosity from about 200 Pa·s to about 16 000 Pa·s, preferably from about 500 Pa·s to about 13 000 Pa·s, more preferably from about 1000 Pa·s to about 12000 Pa·s, even more preferably from about 2900 Pa·s to about 11775 Pa·s, most preferably from about 4500 Pa·s to about 11660 Pa·s, or from about 500 Pa·s to about 7750 Pa·s; or from about 1 500 Pa·s to about 16 000 Pas as measured according to the Carreau Zero Shear Viscosity Method as disclosed herein.

### Ultracentrifugation Method

The Ultracentrifugation Method is used to determine the percent of a structured domain or an opaque structured domain (e.g., a lamellar phase) that is present in a multiphase personal care composition. The method involves the separation of the composition by ultracentrifugation into separate but distinguishable layers. The multiphase personal care composition of the present disclosure can have multiple distinguishable layers (e.g. a structured surfactant layer, and a benefit layer).

A composition is separated by ultracentrifuge into separate but distinguishable layers.

First, dispense about 4 grams of composition into a Beckman Centrifuge Tube (11×60 mm) to fill the tube. Place the centrifuge tubes in an ultracentrifuge (Beckman Model L8-M or equivalent) using a sling rotor and ultracentrifuge using the following conditions: 50,000 rpm, 24 hours, and 40° C.

Measure the relative phase volumes of the phases the composition by measuring the height of each layer using an Electronic Digital Caliper (within 0.01 mm). Layers are identified by those skilled in the art by physical observation techniques paired with chemical identification if needed. For example, the structured surfactant layer is identified by transmission electron microscopically (TEM), polarized light microscopy, and/or X-ray diffraction for the present disclosure as a structured lamellar phase comprising multilamellar vesicles, and the hydrophobic benefit layer is identified by its low moisture content (less than 10% water as measured by Karl Fischer Titration). The total height Hₐ is measured which includes all materials in the ultracentrifuge tube. Next, the height of each layer is measured from the bottom of the centrifuge tube to the top of the layer, and the span of each layer algebraically determined by subtraction. The benefit layer may comprise several layers if the benefit phase has more than one component which may phase splits into liquid and waxy layers, or if there is more than one benefit component. If the benefit phase splits, the sum of the benefit layers measured is the benefit layer height, H_{b}. In some cases, in case of incomplete separation of the benefit phase from the cleansing phase, the resulting emulsion phase is considered to be part of the benefit phase and is included in the measurement of the benefit layer height, H_{b}. Generally, a hydrophobic benefit layer when present, is at the top of the centrifuge tube.

The cleansing phase may comprise several layers or a single layer, He. There may also be a micellar, unstructured, clear isotropic layer which may contain the rheology modifiers at the bottom or next to the bottom of the ultracentrifuge tube. The layers immediately above the isotropic phase generally comprise higher surfactant concentration with higher ordered structures (such as liquid crystals). These structured layers are sometimes opaque to naked eyes, or translucent, or clear. There may be several structured layers present, in which case H_{c} is the sum of the individual structured layers. If any type of polymer-surfactant phase is present, it is considered a structured phase and included in the measurement of H_{c}. The sum of the aqueous phases is Hₛ.

Finally, the structured domain volume ratio is calculated as follows:

$$Structured Domain Volume Ratio = {H}_{c} / {H}_{s} * 100 \%$$

If there is no benefit phase present, use the total height as the surfactant layer height, Hₛ=Hₐ. The Structured Domain Volume Ratio is the Lamellar Phase %.

The personal care composition may have a Structured Domain Volume Ratio of at least about 40%, alternatively at least about 45%, alternatively at least about 50%, alternatively at least about 55%, alternatively at least about 60%, alternatively at least about 65%, alternatively at least about 70%, alternatively at least about 75%, alternatively at least about 80%, alternatively at least about 85%, and alternatively greater than about 90% by volume of the aqueous structured surfactant phase.

### Examples

The following examples further describe the personal care compositions described herein. The examples are given solely for the purpose of illustration and are not to be construed as limitations of the present disclosure, as many variations thereof are possible without departing from the spirit and scope of the disclosure. Where applicable, ingredients are identified by chemical or CTFA name, or otherwise defined below.

The following examples CEx. 1-6 were prepared: The compositions below were prepared by adding water in a mixing vessel. Then, the following ingredients have been with continuously mixing until obtaining a homogenous mixture: cocamidopropyl betaine, sodium benzoate, and sodium salicylate. Then, the following ingredients have been with continuously mixing until obtaining a homogenous mixture: sodium cocoyl alaninate followed by glyceryl caprylate/caprate melted beforehand. For this, glyceryl caprylate/caprate is heated at 40° C. before being added to the batch. The pH is then adjusted by adding citric acid solution (50% active) to pH=5.0±0.2. Keep mixing until homogeneous. The resulting cleansing composition is combined in a stepwise fashion with a perfume and a benefit agent (soybean oil alone or in a mixture of soybean oil at 97 wt. % in a solution with glyceryl monooleate and butylated hydroxytoluene (BHT)) through a Hauschild SpeedMixer™ (Model DAC, 400FV available from FleckTeck, Inc USA) at 1950 rpm for 60 seconds. After forming the personal care compositions, lather, rheology or viscosity and % lamellar phase volume testing are performed after at least 48 hours of equilibration at ambient conditions.

| Ingredients (wt. %) | CEx. 1 | CEx. 2 | CEx. 3 | CEx. 4 |
|---|---|---|---|---|
| Sodium cocoyl alaninate | 18.0 | 18.0 | 18.0 | 14.0 |
| (low salt)¹ | | | | |
| Cocamidopropyl betaine² | 0 | 0 | 0 | 4.0 |
| Glyceryl caprylate/caprate³ | 7.0 | 7.0 | 7.0 | 5.5 |
| Soybean oil⁴ | 1.94 | 3.88 | 9.70 | 1.94 |
| Glyceryl Monooleate | 0.02 | 0.04 | 0.10 | 0.02 |
| BHT (butylated | 0.04 | 0.08 | 0.20 | 0.04 |
| hydroxytoluene) | | | | |
| Citric Acid⁵ | Adjust | Adjust | Adjust | Adjust |
| | pH 5.0 | pH 5.0 | pH 5.0 | pH 5.0 |
| Sodium Benzoate⁶ | 0.45 | 0.45 | 0.45 | 0.45 |
| Sodium salicylate⁷ | 0.40 | 0.40 | 0.40 | 0.40 |
| Perfume | 1.0 | 1.0 | 1.0 | 1.0 |
| Water | q.s. | q.s. | q.s. | q.s. |
| Formula total | 100 | 100 | 100 | 100 |
| Ratio alaninate/GCC | 2.33 | 2.33 | 2.33 | 2.54 |
| Cylinder lather volume | 365 | 335 | 328 | 433 |
| (avg All) (mL) | | | | |
| Carreau Zero shear | 3698 | 4601 | 5041 | 97 |
| Viscosity (Pa · s) | | | | |
| Lamellar Phase Volume (%) | 89% | 89% | 93% | 82% |

When the comparative compositions comprised a level of emulsifying agent here glyceryl caprylate/caprate above 5 wt. %, no acceptable lather volume could be obtained (CEx. 1-3). The rheology property in terms of Carreau zero shear viscosity and % of lamellar phase volume was however acceptable when using 7 wt. % of glyceryl caprylate/caprate and a total surfactant level of 18 wt. %.

When the level of the emulsifying agent here glyceryl caprylate/caprate was decreased to 5.5 wt. % with the same total surfactant level of 18 wt. % with the addition of 4 wt. % of cocamidopropyl betaine, the lather property of the composition has been improved (CEx. 4). However, the improved lather result was to the detriment of the rheology profile since the Carreau Zero Shear Viscosity collapsed.

| Ingredients (wt. %) | C. Ex. 5 | C. Ex. 6 |
|---|---|---|
| Sodium cocoyl alaninate | 14.0 | 14.0 |
| (low salt)¹ | | |
| Cocamidopropyl betaine² | 4.0 | 4.0 |
| Glyceryl caprylate/caprate³ | 2.0 | 2.0 |
| Soybean oil⁴ | 0 | 1.94 |
| Glyceryl Monooleate | 0 | 0.02 |
| BHT | 0 | 0.04 |
| Citric Acid⁵ | Adjust | Adjust |
| | pH 5.0 | pH 5.0 |
| Sodium Benzoate⁶ | 0.45 | 0.45 |
| Sodium salicylate⁷ | 0.40 | 0.40 |
| Perfume | 1.0 | 1.0 |
| Water | q.s. | q.s. |
| Formula total | 100 | 100 |
| Ratio alaninate/GCC | 7 | 7 |
| Cylinder lather volume | 475 | 468 |
| (avg All) (mL) | | |
| Carreau Zero shear | 77 | 70 |
| Viscosity (Pa · s | | |
| Lamellar Phase Volume (%) | 84% | 82% |

When the level of the emulsifying agent here glyceryl caprylate/caprate was decreased to 2 wt. % with the same total surfactant level to 18 wt. % and 4 wt. % of cocamidopropyl betaine (CEx. 5-6), the lather property of the composition was also improved over CEx. 1-3 but still to the detriment of the rheology profile since the Carreau Zero Shear Viscosity collapsed.

The first comparative data showed that the personal care composition needs a cleansing phase comprising an alaninate surfactant as sodium cocoyl alaninate and a zwitterionic surfactant as cocamidopropyl betaine; and a structuring system with at least from about 0.5 wt. % to about 5 wt. % of an emulsifying agent. Here, the one tested was a glyceryl ester and specifically glyceryl caprylate/caprate. The initial first comparative data showed that the composition may have a weight ratio of sodium cocoyl alaninate to glyceryl caprylate/caprate that is greater than about 5:1 to about 15:1.

The following examples Ex. 1-31 were prepared: The compositions below were prepared by adding water in a mixing vessel. Then, the following ingredients have been with continuously mixing until obtaining a homogenous mixture: cocamidopropyl betaine, sodium benzoate, sodium salicylate and the respective rheology modifier was added. Alternatively, a predispersion of the rheology modifier (hydroxypropyl starch phosphate or Acrylates/C10-C30 alkyl acrylates crosspolymer or xanthan gum) in the respective emulsifying agent (glyceryl caprylate/caprate or trideceth-3 or glyceryl laurate or glyceryl stearate) was prepared and added to the batch.

Then, the following ingredients have been added with continuously mixing until obtaining a homogenous mixture: sodium cocoyl alaninate followed by an emulsifying agent (glyceryl caprylate/caprate or trideceth-3 or glyceryl laurate or glyceryl stearate) if not already added in a predispersion as set out above.

Typically, the batches including glyceryl caprylate/caprate or glyceryl laurate are heated to 60-65° C. then cooled to 30-35° C. before pH adjustment. The pH is then adjusted by adding citric acid solution (50% active) to pH=5.0±0.2. Keep mixing until homogeneous. The resulting cleansing composition is combined with a perfume and a benefit agent (soybean oil, shea butter or argan oil; or a mixture of soybean oil at 97 wt. % in a solution with glyceryl monooleate and butylated hydroxytoluene (BHT)) through a Hauschild SpeedMixer™ (Model DAC, 400FV available from FleckTeck, Inc USA) at 1950 rpm for 60 seconds. After forming the personal care compositions, lather and rheology or viscosity testing are performed after at least 48 hours of equilibration at ambient conditions.

| Ingredients (wt. %) | Ex. 1 | Ex. 2 | Ex. 3 | Ex. 4 | Ex. 5 | Ex. 6 |
|---|---|---|---|---|---|---|
| Sodium cocoyl alaninate (low salt)¹ | 14.0 | 14.0 | 14.0 | 14.0 | 14.0 | 14.0 |
| Cocamidopropyl betaine² | 4.0 | 4.0 | 4.0 | 4.0 | 4.0 | 4.0 |
| Glyceryl caprylate/caprate³ | 2.0 | 2.0 | 2.0 | 2.0 | 2.0 | 2.0 |
| Hydroxypropyl starch phosphate⁸ | 1.0 | 1.0 | 0.3 | 0.3 | — | — |
| Xanthan gum⁹ | — | — | — | — | 0.3 | 0.3 |
| Soybean oil⁴ | 1.94 | 9.70 | 1.94 | 9.70 | 1.94 | 9.70 |
| Glyceryl Monooleate | 0.02 | 0.10 | 0.02 | 0.10 | 0.02 | 0.10 |
| Butylated hydroxytoluene (BHT) | 0.04 | 0.20 | 0.04 | 0.20 | 0.04 | 0.20 |
| Citric Acid⁵ | Adjust | Adjust | Adjust | Adjust | Adjust | Adjust |
| | pH 5.0 | pH 5.0 | pH 5.0 | pH 5.0 | pH 5.0 | pH 5.0 |
| Sodium Benzoate⁶ | 0.45 | 0.45 | 0.45 | 0.45 | 0.45 | 0.45 |
| Sodium salicylate⁷ | 0.40 | 0.40 | 0.40 | 0.40 | 0.40 | 0.40 |
| Perfume | 1.0 | 1.0 | 1.0 | 1.0 | 1.0 | 1.0 |
| Water | q.s. | q.s. | q.s. | q.s. | q.s. | q.s. |
| Formula total | 100 | 100 | 100 | 100 | 100 | 100 |
| Ratio alaninate/GCC | 7 | 7 | 7 | 7 | 7 | 7 |
| Cylinder lather volume (avg All) (mL) | 493 | 452 | 478 | 467 | 443 | 440 |
| Carreau Zero shear Viscosity (Pa · s) | 4822 | 10225 | 722 | 4525 | 743 | 2055 |
| Lamellar Phase Volume (%) | 79% | 75% | 80% | 83% | 76% | 83% |

When a rheology modifier is now added such as hydroxypropyl starch phosphate at 1 wt. % and 2 wt. % of glyceryl caprylate/caprate as the emulsifying agent, both lather and rheology properties have been improved. Increasing the benefit agent, namely soybean oil from 1.94 wt. % to 9.70 wt. % has increased the Carreau Zero shear Viscosity to some extent but still while maintaining improved lather properties.

Comparable results have been obtained when using another level of hydroxypropyl starch phosphate at 0.3 wt. % or another type of rheology modifier such as xanthan gum. Even if the viscosity has slightly dropped, the results were acceptable in terms of structuring the cleansing phase, in terms of the viscosity profile with the Lamellar Phase Volume, and Carreau Zero Shear Viscosity.

The first results showed that the structuring system needs to comprise from 0.5 wt. % to 5 wt. % of an emulsifying agent, wherein the emulsifying agent is a glyceryl ester such as but not limited to glyceryl caprylate/caprate; and from 0.01 wt. % to 10 wt. % of a rheology modifier such as but not limited to hydroxypropyl starch phosphate or xanthan gum.

| Ingredients (wt. %) | Ex. 1 | Ex. 2 | Ex. 7 | Ex. 8 | Ex. 9 | Ex. 10 | Ex. 11 | Ex. 12 |
|---|---|---|---|---|---|---|---|---|
| Sodium cocoyl alaninate (low salt)¹ | 14.0 | 14.0 | 14.0 | 14.0 | 14.0 | 14.0 | 14.0 | 14.0 |
| Cocamidopropyl betaine² | 4.0 | 4.0 | 4.0 | 4.0 | 4.0 | 4.0 | 4.0 | 4.0 |
| Glyceryl caprylate/caprate³ | 2.0 | 2.0 | — | — | 2.0 | 2.0 | 2.0 | 2.0 |
| Hydroxypropyl starch phosphate⁸ | 1.0 | 1.0 | 1.0 | 1.0 | 0.5 | 0.5 | 0.3 | 0.3 |
| Trideceth-3¹⁰ | — | — | 2.0 | 2.0 | — | — | — | — |
| Xanthan gum⁹ | — | — | — | — | 0.1 | 0.1 | 0.1 | 0.1 |
| Soybean oil⁴ | 1.94 | 9.70 | 1.94 | 9.70 | 1.94 | 9.70 | 1.94 | 9.70 |
| Glyceryl Monooleate | 0.02 | 0.10 | 0.02 | 0.10 | 0.02 | 0.10 | 0.02 | 0.10 |
| BHT | 0.04 | 0.20 | 0.04 | 0.20 | 0.04 | 0.20 | 0.04 | 0.20 |
| Citric Acid⁵ | Adjust | Adjust | Adjust | Adjust | Adjust | Adjust | Adjust | Adjust |
| | pH 5 | pH 5.0 | pH 5.0 | pH 5.0 | pH 5.0 | pH 5.0 | pH 5.0 | pH 5.0 |
| Sodium Benzoate⁶ | 0.45 | 0.45 | 0.45 | 0.45 | 0.45 | 0.45 | 0.45 | 0.45 |
| Sodium salicylate⁷ | 0.40 | 0.40 | 0.40 | 0.40 | 0.40 | 0.40 | 0.40 | 0.40 |
| Perfume | 1.0 | 1.0 | 1.0 | 1.0 | 1.0 | 1.0 | 1.0 | 1.0 |
| Water | q.s. | q.s. | q.s. | q.s. | q.s. | q.s. | q.s. | q.s. |
| Formula total | 100 | 100 | 100 | 100 | 100 | 100 | 100 | 100 |
| Ratio alaninate/GCC or nonionic | 7 | 7 | 7 | 7 | 7 | 7 | 7 | 7 |
| Cylinder lather volume (avg All) (mL) | 493 | 452 | 395 | 375 | 495 | 470 | 470 | 460 |
| Carreau Zero shear Viscosity (Pa · s) | 4822 | 10225 | 7502 | 12983 | 5602 | 12010 | 648 | 2142 |
| Lamellar Phase Volume (%) | 79% | 75% | 78% | 72% | 84% | 82% | 80% | 83% |

Equivalent results have been obtained when replacing glyceryl caprylate/caprate with another emulsifying agent such as a non-ionic emulsifier having an HLB of from 3.4 to 13.0, i.e. trideceth-3 in Ex. 7-8.

The results showed that the structuring system needs to include from 0.5 wt. % to 5 wt. % of an emulsifying agent, wherein the emulsifying agent is a glyceryl ester such as but not limited to glyceryl caprylate/caprate or a non-ionic emulsifier having an HLB of from 3.4 to 13.0, i.e. trideceth-3 or trideceth-4; and from 0.01 wt. % to 10 wt. % of a rheology modifier such as but not limited to hydroxypropyl starch phosphate.

Another aspect may be related to the combination of two rheology modifiers. In that respect, the personal care composition may comprise a mixture of hydroxypropyl starch phosphate and xanthan gum as shown in Ex. 9-12.

When having a mixture of hydroxypropyl starch phosphate and xanthan gum, the obtained composition could also achieve the desired balance between improved structure and improved lather. Such composition can help for providing effective skin cleansing and deposition of benefit agents.

| Ingredients (wt. %) | Ex. 13 | Ex. 14 | Ex. 15 | Ex. 16 |
|---|---|---|---|---|
| Sodium cocoyl alaninate | 14.0 | 14.0 | 14.0 | 14.0 |
| (low salt)¹ | | | | |
| Cocamidopropyl betaine² | 4.0 | 4.0 | 4.0 | 4.0 |
| Glyceryl caprylate/caprate³ | 2.0 | 2.0 | 2.0 | 2.0 |
| Acrylates/C10-C30 alkyl | 0.1 | 0.1 | 0.3 | 0.3 |
| acrylates crosspolymer¹¹ | | | | |
| (Aqupec SER-300) | | | | |
| Soybean oil⁴ | 1.94 | 9.70 | 1.94 | 9.70 |
| Glyceryl Monooleate | 0.02 | 0.10 | 0.02 | 0.10 |
| BHT | 0.04 | 0.20 | 0.04 | 0.20 |
| Citric Acid⁵ | Adjust | Adjust | Adjust | Adjust |
| | pH 5.0 | pH 5.0 | pH 5.0 | pH 5.0 |
| Sodium Benzoate⁶ | 0.45 | 0.45 | 0.45 | 0.45 |
| Sodium salicylate⁷ | 0.40 | 0.40 | 0.40 | 0.40 |
| Perfume | 1.0 | 1.0 | 1.0 | 1.0 |
| Water | q.s. | q.s. | q.s. | q.s. |
| Formula total | 100 | 100 | 100 | 100 |
| Ratio alaninate/GCC | 7 | 7 | 7 | 7 |
| or nonionic | | | | |
| Cylinder lather volume | 435 | 435 | | |
| (avg All) (mL) | | | | |
| Carreau Zero shear | 3759 | 6381 | | |
| Viscosity (Pa · s) | | | | |
| Lamellar Phase Volume (%) | 83% | 81% | | |

The first results showed that the structuring system needs to include from 0.5 wt. % to 5 wt. % of an emulsifying agent, and from 0.01 wt. % to 10 wt. % of a rheology modifier such as but not limited to hydroxypropyl starch phosphate or xanthan gum.

Also, it has been found that Acrylates/C10-C30 alkyl acrylates crosspolymer could be used as an alternative rheology modifier in Ex. 13-16.

| Ingredients (wt. %) | Ex. 1 | Ex. 2 | Ex. 17 | Ex. 18 | Ex. 19 | Ex. 20 |
|---|---|---|---|---|---|---|
| Sodium cocoyl alaninate (low salt)¹ | 14.0 | 14.0 | 14.0 | 14.0 | 14.0 | 14.0 |
| Cocamidopropyl betaine² | 4.0 | 4.0 | 4.0 | 4.0 | 4.0 | 4.0 |
| Glyceryl caprylate/caprate³ | 2.0 | 2.0 | 1.0 | 1.0 | 2.5 | 2.5 |
| Hydroxypropyl starch phosphate⁸ | 1.0 | 1.0 | 1.0 | 1.0 | 1.0 | 1.0 |
| Soybean oil⁴ | 1.94 | 9.70 | 1.94 | 9.70 | 1.94 | 9.70 |
| Glyceryl Monooleate | 0.02 | 0.10 | 0.02 | 0.10 | 0.02 | 0.10 |
| BHT | 0.04 | 0.20 | 0.04 | 0.20 | 0.04 | 0.20 |
| Citric Acid⁵ | Adjust | Adjust | Adjust | Adjust | Adjust | Adjust |
| | pH 5.0 | pH 5.0 | pH 5.0 | pH 5.0 | pH 5.0 | pH 5.0 |
| Sodium Benzoate⁶ | 0.45 | 0.45 | 0.45 | 0.45 | 0.45 | 0.45 |
| Sodium salicylate⁷ | 0.40 | 0.40 | 0.40 | 0.40 | 0.40 | 0.40 |
| Perfume | 1.0 | 1.0 | 1.0 | 1.0 | 1.0 | 1.0 |
| Water | q.s. | q.s. | q.s. | q.s. | q.s. | q.s. |
| Formula total | 100 | 100 | 100 | 100 | 100 | 100 |
| Ratio alaninate/GCC or nonionic | 7 | 7 | 14 | 14 | 5.6 | 5.6 |
| Cylinder lather volume (avg All) (mL) | 493 | 452 | 495 | 470 | 475 | 475 |
| Carreau Zero shear Viscosity (Pa · s) | 4822 | 10225 | 6118 | 11461 | 5389 | 11660 |
| Lamellar Phase Volume (%) | 79% | 75% | 78% | 75% | 81% | 79% |

Various levels of emulsifying agents have been evaluated, which led to specific weight ratio of the alaninate surfactant to the emulsifying agent (Ex. 17-20). Both lather and Carreau Zero shear viscosity have been maintained.

Another aspect may be related to the combination of two rheology modifiers. In that respect, the personal care composition may comprise a mixture of hydroxypropyl starch phosphate and xanthan gum with other levels of emulsifying agents (See Ex. 21-24).

The personal care composition may comprise from about 0.3 wt. % to about 1.5 wt. % of hydroxypropyl starch phosphate and from about 0.1 wt. % to about 0.5 wt. % of xanthan gum, preferably from about 0.3 wt. % to about 1.0 wt. % of hydroxypropyl starch phosphate and from about 0.1 wt. % to about 0.4 wt. % of xanthan gum. As shown below, the respective compositions could achieve the desired balance between improved structure and improved lather.

Also, soybean oil has been replaced by other benefits agents such as argan oil in Ex. 25 or by shea butter in Ex. 26 resulting in equivalent results in terms of lather or the viscosity profile in terms of both Lamellar Phase Volume, and Carreau Zero Shear Viscosity. Glyceryl Monooleate and butylated hydroxytoluene are optional ingredients, not necessary to provide the benefits disclosed herein.

| Ingredients (wt. %) | Ex. 21 | Ex. 22 | Ex. 23 | Ex. 24 | Ex. 25 | Ex. 26 |
|---|---|---|---|---|---|---|
| Sodium cocoyl alaninate (low salt)¹ | 14.0 | 14.0 | 14.0 | 14.0 | 14.0 | 14.0 |
| Cocamidopropyl betaine² | 4.0 | 4.0 | 4.0 | 4.0 | 4.0 | 4.0 |
| Glyceryl caprylate/caprate³ | 1.0 | 1.0 | 1.0 | 1.0 | 2.0 | 2.0 |
| Hydroxypropyl starch phosphate⁸ | 0.5 | 0.5 | 1.0 | 1.0 | 1.0 | 1.0 |
| Xanthan gum⁹ | 0.1 | 0.1 | 0.3 | 0.3 | — | — |
| Soybean oil⁴ | 1.94 | 9.70 | 1.94 | 9.70 | — | — |
| Glyceryl Monooleate | 0.02 | 0.10 | 0.02 | 0.10 | 0.02 | 0.02 |
| BHT | 0.04 | 0.20 | 0.04 | 0.20 | 0.04 | 0.04 |
| Argan oil¹² | — | — | — | — | 1.94 | — |
| Shea Butter¹³ | — | — | — | — | — | 1.94 |
| Citric Acid⁵ | Adjust | Adjust | Adjust | Adjust | Adjust | Adjust |
| | pH 5.0 | pH 5.0 | pH 5.0 | pH 5.0 | pH 5.0 | pH 5.0 |
| Sodium Benzoate⁶ | 0.45 | 0.45 | 0.45 | 0.45 | 0.45 | 0.45 |
| Sodium salicylate⁷ | 0.40 | 0.40 | 0.40 | 0.40 | 0.40 | 0.40 |
| Perfume | 1.0 | 1.0 | 1.0 | 1.0 | 1.0 | 1.0 |
| Water | q.s. | q.s. | q.s. | q.s. | q.s. | q.s. |
| Formula total | 100 | 100 | 100 | 100 | 100 | 100 |
| Ratio alaninate/GCC or nonionic | 14 | 14 | 14 | 14 | 7.0 | 7.0 |
| Cylinder lather volume (avg All) (mL) | 495 | 455 | 510 | 465 | 500 | 465 |
| Carreau Zero shear Viscosity (Pa · s) | 3811 | 11152 | 7280 | 15612 | 4204 | 4336 |
| Lamellar Phase Volume (%) | 82% | 84% | 71% | 69% | 80% | 80% |

Other glyceryl esters such as glyceryl laurate or glyceryl stearate have been assessed to show comparable results as glyceryl caprylate/caprate (Ex. 27-30). Glyceryl caprylate/caprate appeared to provide an improved structure with an enhanced Carreau Zero Shear viscosity (Ex. 23-24).

| Ingredients (wt. %) | Ex. 27 | Ex. 28 | Ex. 29 | Ex. 30 | Ex. 31 |
|---|---|---|---|---|---|
| Sodium cocoyl alaninate | 14.0 | 14.0 | 14.0 | 14.0 | 14.0 |
| (low salt)¹ | | | | | |
| Cocamidopropyl betaine² | 4.0 | 4.0 | 4.0 | 4.0 | — |
| Cocamidopropyl betaine | — | — | — | — | 4.0 |
| (low salt)^{2a} | | | | | |
| Glyceryl caprylate/ | — | — | — | — | 2.0 |
| caprate³ | | | | | |
| Glyceryl laurate¹⁴ | 1.0 | 1.0 | — | — | — |
| Glyceryl stearate¹⁵ | — | — | 1.0 | 1.0 | — |
| Hydroxypropyl starch | 1.0 | 1.0 | 1.0 | 1.0 | 1.0 |
| phosphate⁸ | | | | | |
| Soybean oil⁴ | 1.94 | 9.70 | 1.94 | 9.70 | 1.94 |
| Glyceryl Monooleate | 0.02 | 0.10 | 0.02 | 0.10 | 0.02 |
| BHT | 0.04 | 0.20 | 0.04 | 0.20 | 0.04 |
| Citric Acid⁵ | Adjust | Adjust | Adjust | Adjust | Adjust |
| | pH 5.0 | pH 5.0 | pH 5.0 | pH 5.0 | pH 5.0 |
| Sodium Benzoate⁶ | 0.45 | 0.45 | 0.45 | 0.45 | 0.45 |
| Sodium salicylate⁷ | 0.40 | 0.40 | 0.40 | 0.40 | 0.40 |
| Perfume | 1.0 | 1.0 | 1.0 | 1.0 | 1.0 |
| Water | q.s. | q.s. | q.s. | q.s. | q.s. |
| Formula total | 100 | 100 | 100 | 100 | 100 |
| Ratio alaninate/GCC | 14 | 14 | 14 | 14 | 14 |
| or nonionic | | | | | |
| Cylinder lather volume | 540 | 510 | 510 | 490 | |
| (avg All) (mL) | | | | | |
| Carreau Zero shear | 3720 | 4039 | 4666 | 4910 | |
| Viscosity (Pa · s) | | | | | |
| Lamellar Phase Volume | 82% | 78% | 77% | 76% | |
| (%) | | | | | |

Ex. 31 is a composition with a low level of sodium chloride content. For this, the anionic surfactant sodium cocoyl alaninate and the zwitterionic surfactant cocamidopropyl betaine have been selected such as containing a relatively low level of sodium chloride. Such composition can help to improve the properties as disclosed herein, especially the lamellar phase volume.

### Definitions of Components

*1 Sodium cocoyl alaninate; Eversoft ACS (contains circa 1 wt. % NaCl), Supplier Sino Lion;
*2 Cocamidopropyl Betaine; SensaFoam™ CK PH 12/MB, Supplier Kensing™, having a 30% active matter and a sodium chloride content of about 5 wt. %.
*2a Cocamidopropyl Betaine; Dehyton® PK 45 having the sodium chloride content removed, Supplier BASF, resulting in about 33.05 wt. % dry residue; and about 0.21 wt. % sodium chloride;
*3 Glyceryl caprylate/caprate; Stepan Mild GCC, Supplier Stepan, containing mono, di, triglycerides, Supplier Stepan Company;
*4 Soybean oil, RBD Soybean oil, Supplier Cargill
*5 Citric acid powder; Supplier Yixing Union Biochemical
*6 Sodium benzoate; Supplier Wuhan Youji Industries
*7 Sodium salicylate; Supplier JQC Huayn Pharmaeutical Co Ltd.
*8 Hydroxypropyl starch phosphate, Structure XL, Supplier Nouryon Chemicals
*9 Xanthan gum, Keltrol 1000, Supplier CPKelco
*10 Trideceth-3, Iconal TDA-3, Supplier BASF Corp.
*11 Acrylates/C10-C30 alkyl acrylates crosspolymer, Aqupec SER-300, Supplier Sumitomo Seika Chemicals
*12 Argan oil, Argania Spinosa Kernel Oil, Supplier Carrubba
*13 Shea Butter, Shebu refined, Supplier Rita
*14 Glyceryl laurate, Monomuls® 90-L 12, Supplier BASF
*15 Glyceyl stearate, Lexemul® 515 MB, Supplier Inolex
q.s.: sufficient quantity

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising at least a cleansing phase and a benefit phase, wherein the composition is substantially free of alkyl sulfate and alkyl ether sulfate type of surfactants;
wherein the cleansing phase comprises an aqueous structured surfactant phase;
wherein the cleansing phase comprises, by weight of the composition:
(a) an alaninate surfactant;
(b) a zwitterionic or amphoteric surfactant;
(c) a structuring system comprising
(i) from about 0.5 wt. % to about 5 wt. % of an emulsifying agent, wherein the emulsifying agent is a glyceryl ester and/or a non-ionic emulsifier having an HLB of from about 3.4 to about 13.0;
wherein the glyceryl ester is selected from glyceryl laurate, glyceryl caprate, glyceryl caprylate, glyceryl caprylate/caprate, glyceryl stearate, and a mixture thereof;
wherein the non-ionic emulsifier having an HLB of from about 3.4 to about 13.0 comprises trideceth-3 or trideceth-4;
(ii) from about 0.01 wt. % to about 10 wt. % of a rheology modifier;
wherein the benefit phase comprises, by weight of the composition: from about 0.1 wt. % to about 50 wt. % of a benefit agent; and
wherein a pH is from about 4.0 to about 5.5.

2. The personal care composition of claim 1, wherein the alaninate surfactant is chosen from sodium cocoyl alaninate, triethylamine cocoyl alaninate, sodium lauroyl alaninate, sodium N-dodecanoyl-1-alaninate, or mixtures thereof.

3. The personal care composition of claim 1, wherein the composition comprises from about 5 wt. % to about 20 wt. % of the alaninate surfactant.

4. The personal care composition of claim 1, wherein the composition comprises from about 5 wt. % to about 20 wt. % of sodium cocoyl alaninate.

5. The personal care composition of claim 1, wherein the composition comprises from about 1 wt. % to about 3 wt. % of the emulsifying agent.

6. The personal care composition of claim 1, wherein a weight ratio of the alaninate surfactant to the emulsifying agent is greater than about 5:1 to about 15:1.

7. The personal care composition of claim 1, wherein a weight ratio of sodium cocoyl alaninate to glyceryl caprylate/caprate is greater than about 5:1 to about 15:1.

8. The personal care composition of claim 1, wherein a weight ratio of sodium cocoyl alaninate to trideceth-3 is greater than about 5:1 to about 15:1.

9. The personal care composition of claim 1, wherein the composition comprises from about 0.1 wt. % to about 5 wt. % of the rheology modifier.

10. The personal care composition of claim 1, wherein the rheology modifier is chosen from sodium polyacrylate, acrylates copolymer, Acrylates/Vinyl Isodecanoate Crosspolymer, Acrylates/C10-30 Alkyl Acrylate Crosspolymer, Acrylates/C10-30 alkyl acrylate crosspolymer having stearyl side chains with less than about 1% Hydrophobic modification, Acrylates/C10-30 alkyl acrylate crosspolymer having octyl side chains with less than about 5% Hydrophobic modification, Ammonium Acryloyldimethyltaurate/Beheneth-25 Methacrylate Crosspolymer, Acrylates/Beheneth-25 Methacrylate Copolymer, Acrylates/Steareth-20 Methacrylate Copolymer, Acrylates/Steareth-20 Methacrylate Crosspolymer, PEG-150/Decyl Alcohol/SMDI Copolymer, PEG-150/stearyl alcohol/SMDI copolymer, hydroxypropyl starch phosphate, distarch phosphate, sodium carboxymethyl starch, starch, Tapioca starch, xanthan gum, gellan gum, carboxymethyl cellulose, carboxymethyl hydroxyethyl cellulose, hydroxypropyl methyl cellulose, sodium alginate, or mixtures thereof.

11. The personal care composition of claim 10, wherein the composition comprises from about 0.01 wt. % to about 10 wt. % of hydroxypropyl starch phosphate.

12. The personal care composition of claim 10, wherein the composition comprises from about 0.3 wt. % to about 1.5 wt. % of hydroxypropyl starch phosphate and from about 0.1 wt. % to about 0.5 wt. % of xanthan gum.

13. The personal care composition of claim 1, wherein the composition comprises from about 0.01 wt. % to about 20 wt. % of the zwitterionic surfactant.

14. The personal care composition of claim 1, wherein the zwitterionic surfactant is chosen from cocamidopropyl betaine, coco-betaine, lauramidopropyl betaine, or mixtures thereof.

15. The personal care composition of claim 1, wherein the composition comprises from about 0.5 wt. % to about 15 wt. % of the benefit agent.

16. The personal care composition of claim 1, wherein the benefit agent is chosen from petrolatum; lanolin; natural waxes, synthetic waxes, volatile organosiloxanes, non-volatile organosiloxanes, lanolin oil, lanolin esters, argan oil, castor oil, soybean oil, derivatized soybean oils, maleated soybean oil, safflower oil, cotton seed oil, corn oil, walnut oil, peanut oil, olive oil, cod liver oil, sweet almond oil, almond oil, avocado oil, palm oil and sesame oil, vegetable oils, sunflower seed oil, and vegetable oil derivatives, coconut oil and derivatized coconut oil, cottonseed oil and derivatized cottonseed oil, jojoba oil, cocoa butter, shea butter, groundnut oil, camellia oil, beauty-leaf oil, rapeseed oil, coconut kernel, coriander oil, marrow oil, wheat germ oil, jojoba oil or liquid jojoba wax, linseed oil, macadamia oil, corn germ oil, hazelnut oil, walnut oil, vemonia oil, apricot kernel oil, olive oil, evening-primrose oil, palm oil, passion flower oil, grapeseed oil, rose oil, castor oil, rye oil, sesame oil, rice bran oil, camelina oil, soybean oil, sunflower oil, pracaxi oil, babassu oil, mongongo oil, marula oil, arara oil, shea butter oil, Brazil nut oil, caprylic/capric acid triglycerides or mixtures thereof.

17. The personal care composition of claim 1, wherein the composition is substantially free of one or more inorganic electrolytes; or substantially free of sodium chloride.

18. The personal care composition of claim 1, wherein the composition comprises a lather volume from about 375 mL to about 575 mL, as measured according to the Cylinder Method as disclosed herein.

19. The personal care composition of claim 1, wherein the composition comprises a Carreau Zero Shear Viscosity from about 200 Pa·s to about 16 000 Pa·s, as measured according to the Carreau Zero Shear Viscosity Method as disclosed herein.

20. A method of making a liquid personal care composition having an acceptable lather or providing a substantial amount of foam or lather when agitated or mixed with water comprising the step of forming a personal care composition of claim 1.
